(19) United States
(12) Patent Application Publication
(10) Pub. No.: US 2024/0299388 A1
(43) Pub. Date: Sep. 12, 2024

(54) ERK1/2 AND SHP2 INHIBITORS COMBINATION THERAPY

(71) Applicant: Erasca, Inc., San Diego, CA (US)

(72) Inventors: Robert Field SHOEMAKER, San Diego, CA (US); Erin Denise LEW, San Diego, CA (US); Wei LIN, San Diego, CA (US); Leslie Harris BRAIL, San Diego, CA (US); Leenus MARTIN, San Diego, CA (US); Jingchuan ZHANG, San Diego, CA (US); Joanne OH, San Diego, CA (US)

(21) Appl. No.: 18/572,404

(22) PCT Filed: Jun. 23, 2022
(86) PCT No.: PCT/US2022/034754

§ 371 Date: Dec. 20, 2023

Related U.S. Application Data
(60) Provisional application No. 63/321,615, filed on Mar. 18, 2022.
(60) Provisional application No. 63/280,521, filed on Nov. 17, 2021.
(60) Provisional application No. 63/277,550, filed on Nov. 9, 2021.
(60) Provisional application No. 63/214,769, filed on Jun. 24, 2021.

(51) Int. Cl. A61K 31/506; A61K 31/497; A61K 31/519; A61K 31/5383; A61K 39/395; A61K 45/06; A61P 35/00

(52) U.S. Cl. CPC A61K 31/506; A61K 31/497; A61K 31/519; A61K 31/5383; A61K 39/3955; A61K 45/06; A61P 35/00

(57) ABSTRACT

The present disclosure relates generally to the use an ERK1/2 inhibitor in combination with a SHP2 inhibitor for treating cancer, specifically solid tumors.

### CROSS-REFERENCE

This application claims the benefit of U.S. Provisional Application Ser. No. 63/214,769 filed Jun. 24, 2021, U.S. Provisional Application Ser. No. 63/277,550 filed Nov. 9, 2021, U.S. Provisional Application Ser. No. 63/280,521 filed Nov. 17, 2021, and U.S. Provisional Application Ser. No. 63/321,615 filed Mar. 18, 2022, which are hereby incorporated by reference in their entirety.

### BACKGROUND

ERK1 and ERK2 (collectively “ERK1/2”) are related protein-serine/threonine kinases that participate in, amongst others, the Ras-Raf-MEK-ERK signal transduction pathway, which is sometimes denoted as the mitogen-activated protein kinase (MAPK) pathway. This pathway is thought to play a central role in regulating a number of fundamental cellular processes including one or more of cell proliferation, survival, adhesion, cycle progression, migration, differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

### SUMMARY

ERK1/2 appear to be activated by MEK through phosphorylation of both a threonine and a tyrosine residue, namely at Tyr204/187 and Thr202/185. Once activated, ERK1/2 catalyze the phosphorylation of serine/threonine residues of more than 100 substrates and activate both cytosolic and nuclear proteins that are linked to cell growth, proliferation, survival, angiogenesis and differentiation, all hallmarks of the cancer phenotype. Thus, it may be beneficial to target ERK 1 and ERK 2 to develop and use ERK1/2 inhibitors as a way to inhibit tumor growth.

Furthermore, an ERK inhibitor may have utility in combination with other kinase, for example MAPK, inhibitors. Recently, researchers reported that dual inhibition of MEK and ERK by small molecule inhibitors was synergistic and acted to overcome acquired resistance to MEK inhibitors. See Hatzivassiliou et al., ERK Inhibition Overcomes Acquired Resistance to MEK Inhibition, Mol. Cancer Ther. 2012, 11, 1143-1154.

In addition to ERK1/2, SHP2 also operates upstream of the RAS pathway. SHP2 is a protein tyrosine phosphatase and a key positive regulator of the growth signals from the RTK growth factor receptors to the intracellular signaling pathways (including RAS/MAPK and PI3K) that promote growth and survival of normal cells and cancer cells. As such, SHP2 is a convergent node for upstream RTK signaling: activated SHP2 upregulates (“turns up”) the positive signals and downregulates (“turns down”) the negative signals in the signaling cascades. SHP2 also serves as a central node in relaying the growth and survival signals from RTKs such as EGFR and FLT3 to RAS/MAPK and other intracellular pathways. SHP2 is an attractive target because SHP2 inhibition ubiquitously blocks the growth signals from multiple RTKs, preventing cancer cells from bypassing the blockade on a specific RTK (e.g., EGFR inhibitor) through activation of other RTK growth factor receptors (e.g., MET).

The opportunity to target signal transduction pathways from multiple angles and potentially ameliorate feedback loops upstream of Ras via ERK1/2 and SHP2 provides opportunities for developing methods that employ combination therapies.

The present embodiments disclosed herein generally relate to compositions and methods related to combination therapies to treat cancer utilizing an ERK1/2 inhibitor in conjunction with a SHP2 inhibitor while providing an unexpected degree of synergy.

Disclosed herein is a method of treating cancer in a subject in need thereof, the method comprising: administering to the subject in need thereof a therapeutically effective amount of
(i) compound 1:

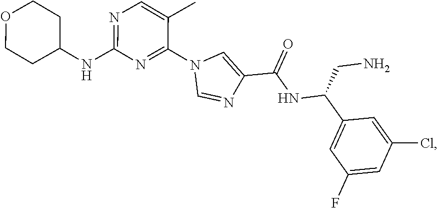 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a SHP2 inhibitor.

In some embodiments, the SHP2 inhibitor is sodium stibogluconate, RMC-4550, NSC87877, SPI-112, TN0155, IACS-13909, SHP099 HCl, or compound 2:

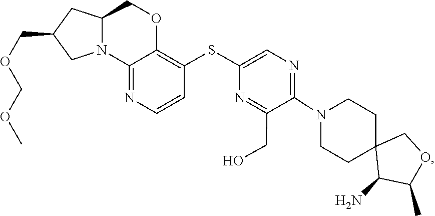 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Also disclosed herein is a method of treating cancer in a subject in need thereof, the method comprising: administering to the subject in need thereof a therapeutically effective amount of
(i) compound 1:

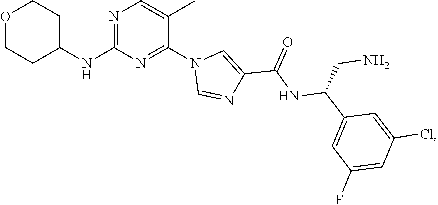 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) compound 2:

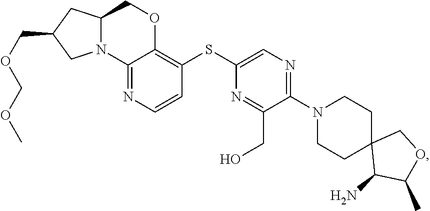 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Also disclosed herein is a method of treating cancer in a subject in need thereof, the method comprising: administering to the subject in need thereof a therapeutically effective amount of
(i) compound 1:

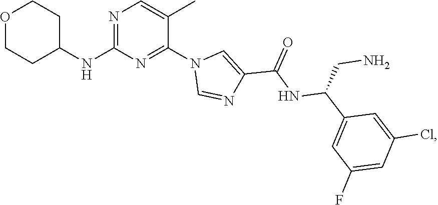 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

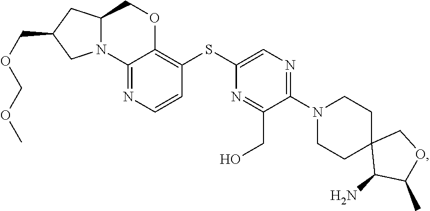 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) osimertinib.

In some embodiments, osimertinib is administered in an amount that is about 80 mg/day.

Also disclosed herein is a method of treating cancer in a subject in need thereof, the method comprising: administering to the subject in need thereof a therapeutically effective amount of
(i) compound 1:

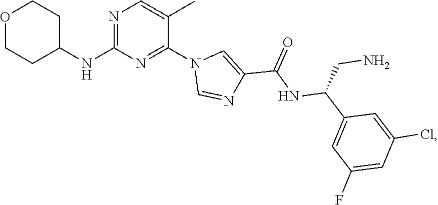 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

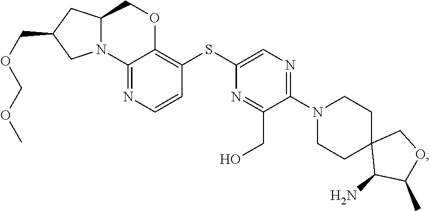 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) cetuximab.

In some embodiments, cetuximab is administered at 500 mg/m² once every two weeks, 400 mg/m² once every two weeks, or 300 mg/m² once every two weeks.

Also disclosed herein is a method of treating cancer in a subject in need thereof, the method comprising: administering to the subject in need thereof a therapeutically effective amount of
(i) compound 1:

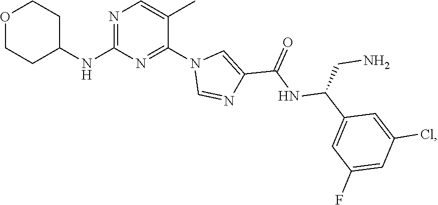 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

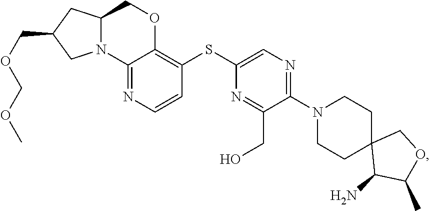 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof;
(iii) encorafenib; and
(iv) osimertinib.

In some embodiments, encorafenib is administered in an amount that is between about 100 mg/day and about 500 mg/day.

In some embodiments, encorafenib is administered in an amount that is about 450 mg/day, 300 mg/day, 225 mg/day, or 150 mg/day.

In some embodiments, osimertinib is administered at about 80 mg/day.

Also disclosed herein is a method of treating cancer in a subject in need thereof, the method comprising: administering to the subject in need thereof a therapeutically effective amount of
(i) compound 1:

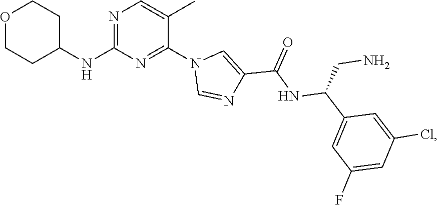 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

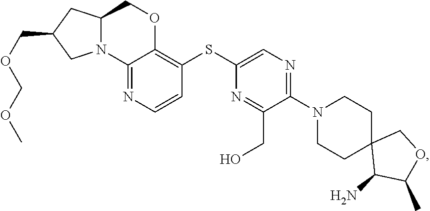 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof;
(iii) encorafenib; and
(iv) cetuximab.

In some embodiments, encorafenib is administered in an amount that is between about 100 mg/day and about 500 mg/day.

In some embodiments, encorafenib is administered in an amount that is about 450 mg/day, about 300 mg/day, about 225 mg/day, or about 150 mg/day.

In some embodiments, cetuximab is administered at 500 mg/m² once every two weeks, 400 mg/m² once every two weeks, or 300 mg/m² once every two weeks.

Also disclosed herein is a method of treating cancer in a subject in need thereof, the method comprising: administering to the subject in need thereof a therapeutically effective amount of
(i) compound 1:

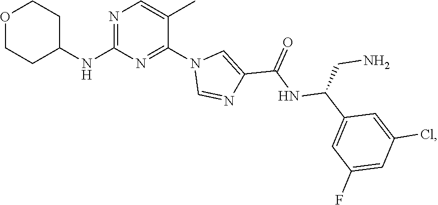 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

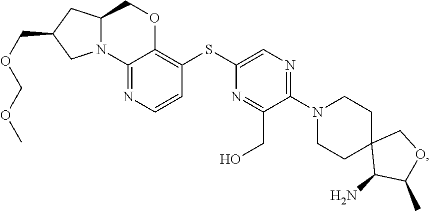 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) gilteritinib.

In some embodiments, gilteritinib is administered in an amount that is about 120 mg/day.

Also disclosed herein is a method of treating cancer in a subject in need thereof, the method comprising: administering to the subject in need thereof a therapeutically effective amount of
(i) compound 1:

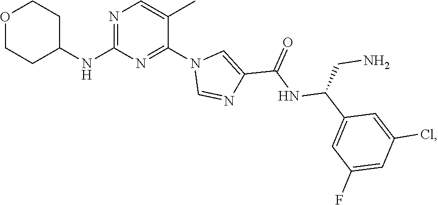 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

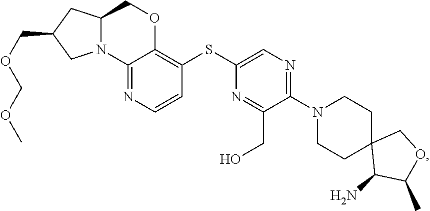 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) sotorasib.

In some embodiments, sotorasib is administered in an amount that is about 960 mg/day.

Also disclosed herein is a method of treating cancer in a subject in need thereof, the method comprising: administering to the subject in need thereof a therapeutically effective amount of
(i) compound 1:

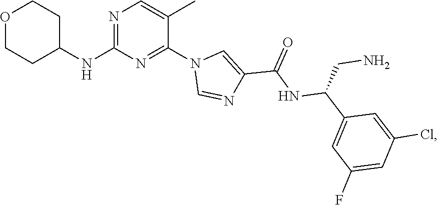 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

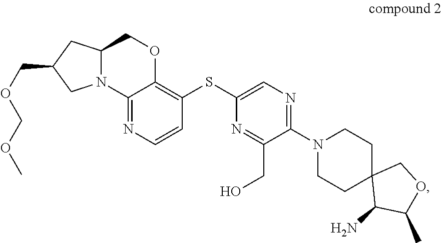 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) adagrasib.

In some embodiments, adagrasib is administered in an amount that is about 1200 mg/day.

Also disclosed herein is a method of treating cancer in a subject in need thereof, the method comprising: administering to the subject in need thereof a therapeutically effective amount of
(i) compound 1:

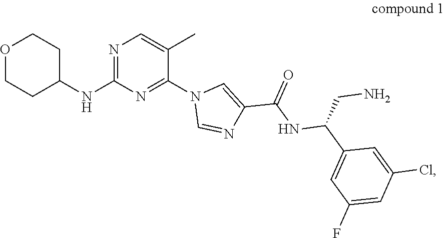 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

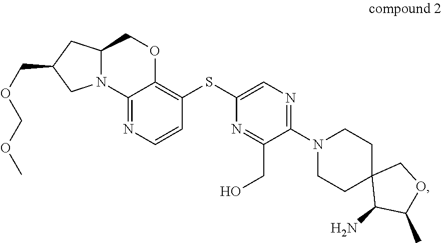 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) palbociclib.

In some embodiments, palbociclib is administered in an amount that is between about 50 mg/day and about 500 mg/day.

In some embodiments, palbociclib is administered in an amount that is about 75 mg/day, about 100 mg/day, about 125 mg/day, or about 150 mg/day.

In some embodiments, palbociclib is administered in an amount that is between about 50 mg once a week and about 650 mg once a week.

In some embodiments, palbociclib is administered in an amount that is about 200 mg once a week, 300 mg once a week, 400 mg once a week, 500 mg once a week, or 600 mg once a week.

In some embodiments, compound 2, or a pharmaceutically acceptable salt thereof, is administered orally.

In some embodiments, compound 2, or a pharmaceutically acceptable salt thereof, is administered in an amount that is between about 1 mg/day and about 500 mg/day.

In some embodiments, compound 2, or a pharmaceutically acceptable salt thereof, is administered in an amount that is between about 20 mg/day and about 400 mg/day.

In some embodiments, compound 2, or a pharmaceutically acceptable salt thereof, is administered in an amount that is between about 30 mg/day and about 300 mg/day.

In some embodiments, compound 2, or a pharmaceutically acceptable salt thereof, is administered QD or BID for 2 weeks on and 1 week off (21 day schedule).

In some embodiments, compound 2, or a pharmaceutically acceptable salt thereof, is administered QD or BID for 3 weeks on and 1 week off (28 day schedule).

In some embodiments, compound 2, or a pharmaceutically acceptable salt thereof, is administered QD or BID three times a week (D1D3D5 TIW) e.g., Day 1, Day 3, and Day 5.

In some embodiments, compound 2, or a pharmaceutically acceptable salt thereof, is administered twice a day/twice a week e.g., Day 1 and Day 2 (BID-D1D2-BIW).

In some embodiments, compound 2, or a pharmaceutically acceptable salt thereof, is administered once a day (QD) continuous dosing at a dose of 20 mg/day to 60 mg/day, 40 mg/day, or 60 mg/day.

In some embodiments, compound 2, or a pharmaceutically acceptable salt thereof, is administered twice a day (BID) continuous dosing at a dose of 20 mg/day to 80 mg/day.

In some embodiments, compound 2, or a pharmaceutically acceptable salt thereof, is administered twice a day (BID) continuous dosing at a dose of 10 mg/day to 100 mg/day.

In some embodiments, the pharmaceutically acceptable salt of compound 1 is the mandelic acid salt.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G13D KRAS mutation. In some embodiments, the cancer has a Q16H KRAS mutation. In some embodiments, the cancer has a Q16K KRAS mutation. In some embodiments, the cancer has a Q61R NRAS mutation.

In some embodiments, the cancer is a BRAF V600E or V600K mutant tumor.

In some embodiments, the cancer is a MAPKm/MAPKi-naïve pancreatic cancer or PDAC.

In some embodiments, the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

In some embodiments, the cancer comprises one or more EGFR mutation selected from the group consisting of L858R, exon 19 deletion, and T790M.

In some embodiments, the cancer is a liquid tumor.

In some embodiments, the liquid tumor is leukemia.

In some embodiments, the leukemia is acute myeloid leukemia (AML).

In some embodiments, the AML is relapsed and/or refractory AML.

In some embodiments, the AML is a FLT3 mutant AML.

In some embodiments, the cancer is a solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G12A mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC. In some embodiments, the NSCLC is a NRAS Q61R mutant NSCLC. In some embodiments, the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61RNSCLC.

In some embodiments, the cancer is pancreatic cancer.

In some embodiments, the cancer is a MAPKm/MAPKi-naïve pancreatic cancer.

In some embodiments, the pancreatic cancer is pancreatic ductal adenocarcinoma (PDAC).

In some embodiments, the cancer is melanoma.

In some embodiments, the melanoma has an NF1 Loss of Function (NF1-LoF) mutation.

In some embodiments, the melanoma is a BRAF V600E or V600K mutant tumor.

In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC. In some embodiments, the CRC is a KRAS G12C mutant CRC. In some embodiments, the CRC is a KRAS G12D mutant CRC.

In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is NRAS Q61R mutant CRC.

In some embodiments, the cancer is esophageal cancer.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 150 mg/day, about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 1, or a pharmaceutically acceptable salt thereof, is administered in an amount that is about 250 mg/day.

In some embodiments, compound 1, or a pharmaceutically acceptable salt thereof, is administered once a day (QD).

In some embodiments, compound 1, or a pharmaceutically acceptable salt thereof, is administered twice a day (BID).

In some embodiments, compound 1, or a pharmaceutically acceptable salt thereof, is administered three times a day (TID).

In some embodiments, compound 1, or a pharmaceutically acceptable salt thereof, is administered once a week.

In some embodiments, compound 1, or a pharmaceutically acceptable salt thereof, is administered twice a week.

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered for at least one 28-day cycle.

In some embodiments, compound 1, or a pharmaceutically acceptable salt thereof, is administered on day 1, day 8, day 15, and day 22 of a 28-day cycle.

In some embodiments, compound 1, or a pharmaceutically acceptable salt thereof, is administered on day 1, day 8, day 15 of a 28-day cycle.

In some embodiments, compound 1, or a pharmaceutically acceptable salt thereof, is administered orally.

In some embodiments, the method further comprises administering an additional MAPK pathway inhibitor. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. In some embodiments, the additional MAPK pathway inhibitor is a KRAS inhibitor. In some embodiments, the additional MAPK pathway inhibitor is a BRAF inhibitor. In some embodiments, the additional MAPK pathway inhibitor is an EGFR inhibitor. In some embodiments, the additional MAPK pathway inhibitor is a CDK4/6.

In some embodiments, the additional MAPK pathway inhibitor is a FLT3 inhibitor. In some embodiments, the additional MAPK pathway inhibitor is adagrasib, afatinib, ASTX029, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, panitumumab, selumetinib, sotorasib, trametinib, ulixertinib, vandetanib, or vemurafenib. In some embodiments, the additional MAPK pathway inhibitor is adagrasib. In some embodiments, the additional MAPK pathway inhibitor is cetuximab. In some embodiments, the additional MAPK pathway inhibitor is dabrafenib. In some embodiments, the additional MAPK pathway inhibitor is encorafenib. In some embodiments, the additional MAPK pathway inhibitor is gilteritinib. In some embodiments, the additional MAPK pathway inhibitor is palbociclib. In some embodiments, the additional MAPK pathway inhibitor is panitumumab.

In some embodiments, the additional MAPK pathway inhibitor is sotorasib.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A shows in vivo data for compound 1+compound 2 in mutant RAS CDX model (NCI-H441).

FIG. 1B shows in vivo data for compound 1+compound 2 in mutant RAS CDX model (NCI-H2009).

FIG. 2A shows combination benefit of compound 1+compound 2 in vitro in a KRAS G12V mutant NSCLC Cell line, NCI-H441 in a14-day clonogenic assay.

FIG. 2B shows combination benefit of compound 1+compound 2 in vitro in a KRAS G12D mutant NSCLC cell line, Gp2D, in a 14-day clonogenic assay.

FIG. 3A shows combination benefit of compound 1+compound 2 in vitro in a KRAS G12D mutant pancreatic cell line, Panc 04.03, in a 14-day clonogenic assay.

FIG. 3B shows combination benefit of compound 1+compound 2 in vitro in a KRAS G12D mutant PDAC cell line, HPAC, in a 14-day clonogenic assay.

FIG. 4 shows combination benefit of compound 1+compound 2 in vivo in a KRAS G12D mutant PDAC PDX model, PAN092.

FIG. 5 shows combination benefit of compound 1+compound 2 in vivo in a KRAS G12D mutant PDAC PDX model PAN026.

FIG. 6 shows combination benefit of compound 1+compound 2 in vivo in a NF1 LoF mutant melanoma CDX model, MeWo.

FIG. 7 shows combination benefit of compound 1+compound 2 in vivo in a KRAS G13D mutant CRC CDX model, LoVo.

FIG. 8 shows combination benefit of compound 1+compound 2 in vitro in KRAS G12V mutant PDAC cell line, Capan-2, in a 14-day clonogenic assay.

FIG. 9 shows combination benefit of compound 1+compound 2 in vitro in KRAS G12D mutant PDAC cell line, Panc 10.05, in 14-day clonogenic assay.

FIG. 10 shows combination benefit of compound 1+compound 2 in vitro in KRAS G12V mutant PDAC cell line, Panc 1, in 14-day clonogenic assay.

### DETAILED DESCRIPTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent, or improve an unwanted condition or disease of a patient. In some embodiments, a therapeutic agent such as a compound 1 is directed to the treatment and/or the amelioration of cancers.

“Administering,” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable,” it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

### Compound 1

Disclosed herein is (S)—N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide:

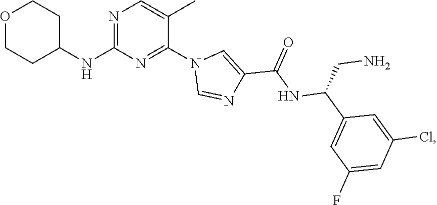 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

In some embodiments, the salt of compound 1 is the mandelic acid salt. In some embodiments, the salt of compound 1 is the benzenesulfonic acid salt. In some embodiments, the salt of compound 1 is the hydrochloride salt. In some embodiments, the salt of compound 1 is the p-toluenesulfonic acid salt.

In some embodiments, the salt of compound 1 is the benzenesulfonic acid salt.

### SHP2 Inhibitor

SHP2 plays important roles in fundamental cellular functions including proliferation, differentiation, cell cycle maintenance and motility, and regulates multiple intracellular signaling pathways in response to wide range of growth factors, cytokines, and hormones. Cell signaling processes in which SHP2 participates include MAPK, PI3K and JAK pathways. SHP2 inhibitors have the potential to attenuate upstream RTK signaling that often drives oncogenic signaling and adaptive tumor escape globally, and to become a broad-spectrum anticancer drug.

In some embodiments, the SHP2 inhibitor is Sodium stibogluconate, RMC-4550, NSC87877, SPI-112, TNO155, IACS-13909, GDC01971, or SHP099 HCl.

In some embodiments, the SHP2 inhibitor is compound 2.

### Compound 2

Disclosed herein is (3-((3S,4S)-4-amino-3-methyl-2-oxa-8-azaspiro[4.5]decan-8-yl)-6-(((6aS,8S)-8-((methoxymethoxy)methyl)-6a,7,8,9-tetrahydro-6H-pyrido[3,2-b]pyrrolo[1,2-d][1,4]oxazin-4-yl)thio)pyrazin-2-yl)methanol:

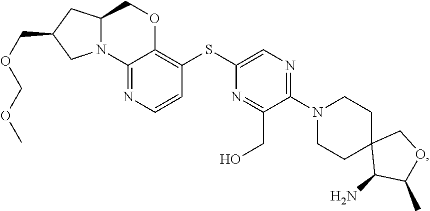 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

### EGFR Inhibitors

EGFR inhibitors are agents that bind to EGFR and slow down or stop cell growth, and can be classified as either tyrosine kinase inhibitors (TKI) or monoclonal antibodies.

TKIs are inhibitors that bind to the tyrosine kinase domain in the epidermal growth factor receptor and stop the activity of the EGFR. Examples include, without limitation, afatinib, dacomitinib, erlotinib, gefitinib, lapatinib, lazertinib, lifirafenib, mobocertinib, nazartinib, neratinib, osimertinib, and vandetanib.

Monoclonal antibody inhibitors are agents that bind to the extracellular component of the EGFR and prevent epidermal growth factor from binding to its own receptor, thus preventing cell division. Examples include, without limitation amivantamab, cetuximab, mirzotamab, clezutoclax, nimotuzumab, and necitumumab.

In some embodiments, the EGFR inhibitor is afatinib, amivantamab, canertinib, cetuximab, dacomitinib, daphnetin, erlotinib, gefitinib, icotinib, lapatinib, lazertinib, lifirafenib, mirzotamab clezutoclax, mobocertinib, nazartinib, necitumumab, neratinib, osimertinib, panitumamab, pelitinib, poziotinib, tivozanib, rociletinib, sapitinib, vandetanib, or varlitinib.

In some embodiments, the EGFR inhibitor is AC480, AEE788, AG-1478, AG-18, AG-490, AST-1306, AV-412, AZ5104, AZD3759, BIBX 1382, CGP-52411, CL-387785, CNX-2006, CUDC-101, OSI-420, PD153035 HCl, PD168393, TAK-285, Tyrphostin 9, Tyrphostin AG 183, WHI-P154, WHI-P180, WZ3146, or WZ4002.

In some embodiments, the EGFR inhibitor is a small molecule inhibitor. In some embodiments, the EGFR inhibitor is osimertinib. In some embodiments, the EGFR inhibitor is cetuximab. In some embodiments, the EGFR inhibitor is afatinib. In some embodiments, the EGFR inhibitor is dacomitinib.

In some embodiments, the EGFR inhibitor is erlotinib. In some embodiments, the EGFR inhibitor is gefitinib. In some embodiments, the EGFR inhibitor is lapatinib. In some embodiments, the EGFR inhibitor is lazertinib. In some embodiments, the EGFR inhibitor is lifirafenib. In some embodiments, the EGFR inhibitor is mobocertinib. In some embodiments, the EGFR inhibitor is nazartinib. In some embodiments, the EGFR inhibitor is neratinib. In some embodiments, the EGFR inhibitor is vandetanib.

In some embodiments, the EGFR inhibitor is not an anti-EGFR antibody inhibitor.

### Osimertinib

Osimertinib

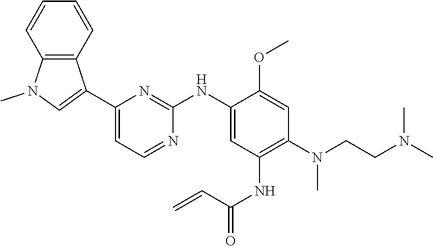

is a small molecule EGFR tyrosine kinase inhibitor and is used to treat locally advanced or metastatic NSCLC. It was approved by the FDA in November 2015 for the specific treatments of metastatic NSCLC with EGFR exon 19 deletions or exon 21 L858R mutations and EGFR T790M mutation-positive NSCLC. Osimertinib is sold as Tagrisso® by AstraZeneca.

### Cetuximab

Cetuximab is a chimeric monoclonal antibody EGFR inhibitor used for the treatment of metastatic CRC and head and neck cancer. In July 2009, cetuximab was approved by the FDA for treatment of colon cancer with wild-type KRAS. Cetuximab is sold as Erbitux® by Eli Lilly and Company.

### BRAF Inhibitors

BRAF inhibitors selectively target BRAF kinase and thus interfere with the MAPK signaling pathway that regulates the proliferation and survival of melanoma cells. BRAF inhibitors also have beneficial effects on the tumor microenvironment and anti-tumor immune response in BRAF-mutant melanoma, thus exerting immunomodulatory effects on the MAPK pathways and promote recognition of the tumor cells by the immune system and enhance anti-tumor T-cell response.

In some embodiments, the BRAF inhibitor is Encorafenib.

In some embodiments, the BRAF inhibitor is Dabrafenib.

### Encorafenib

Encorafenib

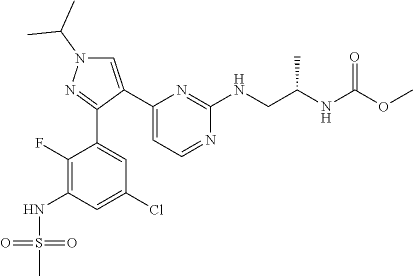 [SMILES: COC(=O)N[C@@H](C)CNc1nccc(-c2cn(C(C)C)nc2-c2cc(Cl)cc(NS(C)(=O)=O)c2F)n1]

is a drug for the treatment of certain melanomas. It is a small molecule BRAF inhibitor that targets key enzymes in the MAPK signaling pathway. This pathway occurs in many different cancers including melanoma and colorectal cancers. In June 2018, it was approved by the FDA in combination with binimetinib for the treatment of patients with unresectable or metastatic BRAF V600E or V600K mutation-positive melanoma. Encorafenib is sold as Braftovi® by Pfizer.

### Dabrafenib

Dabrafenib

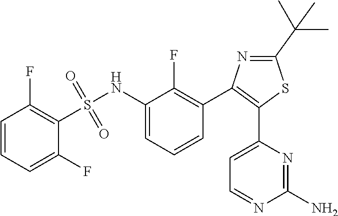 [SMILES: CC(C)(C)c1nc(-c2cccc(NS(=O)(=O)c3c(F)cccc3F)c2F)c(-c2ccnc(N)n2)s1]

is a medication for the treatment of cancers associated with a mutated version of the gene BRAF. Dabrafenib acts as an inhibitor of the associated enzyme B-Raf, which plays a role in the regulation of cell growth. Dabrafenib has clinical activity with a manageable safety profile in clinical trials of phase 1 and 2 in patients with BRAF (V600)-mutated metastatic melanoma. Dabrafenib is sold as Tafinlar® by Novartis.

### CDK4/6Inhibitors

CDK4/6 inhibitors act at the G-to-S cell cycle checkpoint, which is tightly controlled by the D-type cyclins, CDK4 and CDK6. When CDK4 and CDK6 are activated by D-type cyclins, they phosphorylate the retinoblastoma-associated protein (pRb), which releases pRb's suppression of E2F transcription factor family and allow the cell to proceed through cell cycle. In HR+ cancer, cyclin D overexpression is common and loss of pRb is rare, making the G-to-S checkpoint an ideal therapeutic agent.

In some embodiments, the CDK4/6 inhibitor is palbociclib, ribociclib, abemaciclib, FCN-437c, or alvociclib.

In some embodiments, the CDK4/6 inhibitor palbociclib.

### Palbociclib

Palbociclib

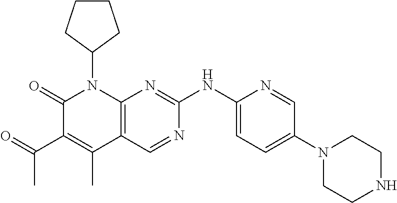 [SMILES: CC(=O)c1c(C)c2cnc(Nc3ccc(N4CCNCC4)cn3)nc2n(C2CCCC2)c1=O]

is a kinase inhibitor used for the treatment of HR+/HER2−advanced or metastatic breast cancer. Palbociclib is sold as Ibrance® by Pfizer.

### FLT3Inhibitors

FLT 3 inhibitors are tyrosine kinase inhibitors that compete for the ATP binding site in the active domain of the kinase, and thus prevent phosphorylation of the protein and decrease the protein's activity.

Type I inhibitors bind to the ATP-binding site when the receptor is active, while type II inhibitors interact with a hydrophobic region immediately adjacent to the ATP-binding site, which is accessible when the receptor is in its inactive conformation. Type I inhibitors include sunitinib, lestaurtinib, midostaurin, crenolanib and gilteritinib, while type II inhibitors include sorafenib, quizartinib and ponatinib.

In some embodiments, the FLT3 inhibitor is crenolanib, gilteritinib, ibrutinib, lestaurtinib, midostaurin, ponatinib, quizartinib, sorafenib, sunitinib, or tandutinib. In some embodiments, the FLT3 inhibitor is crenolanib. In some embodiments, the FLT3 inhibitor is gilteritinib. Gilteritinib is sold under the brand name Xospata® by Astellas Pharma US, Inc.. In some embodiments, the FLT3 inhibitor is ibrutinib. In some embodiments, the FLT3 inhibitor is lestaurtinib. In some embodiments, the FLT3 inhibitor is midostaurin. In some embodiments, the FLT3 inhibitor is ponatinib. In some embodiments, the FLT3 inhibitor is quizartinib. In some embodiments, the FLT3 inhibitor is sorafenib. In some embodiments, the FLT3 inhibitor is sunitinib. In some embodiments, the FLT3 inhibitor is tandutinib. In some embodiments, the FLT3 inhibitor is ibrutinib, ponatinib, quizartinib, crenolanib, or gilteritinib. In some embodiments, the FLT3 inhibitor is sorafenib, lestaurtinib, midostaurin, sunitinib, or tandutinib. In some embodiments, a combination using two or more of the FLT3 inhibitor enumerated above can be combined, such as combining a type I FLT3 inhibitor with a type II FLT3 inhibitor. As an example, gilteritinib can be combined with quizartinib.

### KRAS G12C Inhibitors

KRAS is a key regulator of signaling pathway responsible for cell proliferation, differentiation, and survival. KRAS is the most frequently mutated oncogene in human cancer and mutations in KRAS can results in continuous cellular proliferation and cancer development. The G12C mutation is a single point mutation with a glycine-to-cysteine substitution at codon 12. This substitution favors the activated state of KRAS, amplifying signaling pathways that lead to oncogenesis.

### Sotorasib

Sotorasib

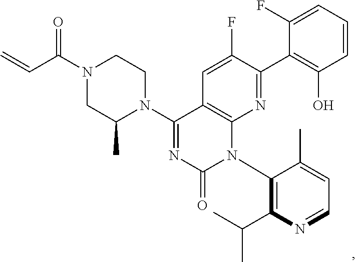

sold under the brand names Lumakras® and Lumykras®, is sold by Amgen and is an anti-cancer medication used to treat non-small-cell lung cancer (NSCLC). It targets a specific mutation, G12C, in the protein K-Ras encoded by gene KRAS which is responsible for various forms of cancer. Sotorasib is an inhibitor of the RAS GTPase family.

Sotorasib is the first approved targeted therapy for tumors with any KRAS mutation, which accounts for approximately 25% of mutations in non-small cell lung cancers. KRAS G12C mutations occur in about 13% of patients with non-small cell lung cancers.

In May 2021, sotorasib was approved by the FDA for the treatment of KRAS G12C mutated NSCLC.

### Combinations

Disclosed herein is a method of treating cancer in a subject in need thereof, the method comprising: administering to the subject in need thereof a therapeutically effective amount of
(i) compound 1:

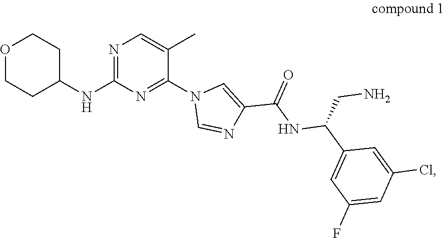 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) a SHP2 inhibitor.

In some embodiments, the SHP2 inhibitor is compound 2:

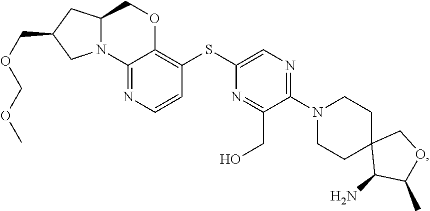 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable alt thereof.

Disclosed herein is a method of treating cancer in a subject in need thereof, the method comprising: administering to the subject in need thereof a therapeutically effective amount of
(i) compound 1:

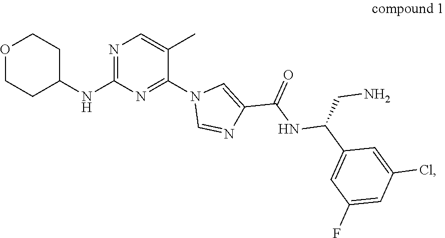 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) compound 2:

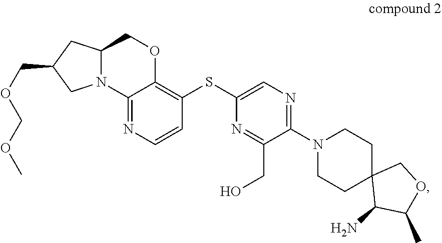 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Disclosed herein is a method of treating cancer in a subject in need thereof, the method comprising: administering to the subject in need thereof a therapeutically effective amount of
(i) compound 1:

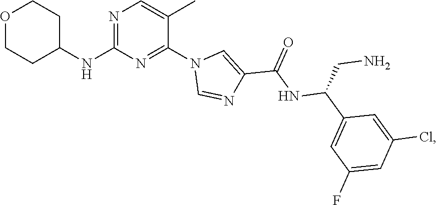 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

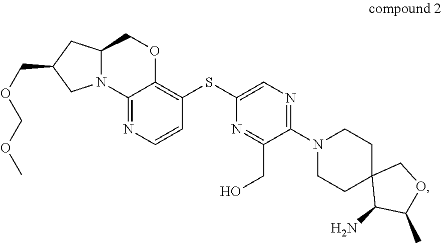 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) an EGFR inhibitor.

Disclosed herein is a method of treating cancer in a subject in need thereof, the method comprising: administering to the subject in need thereof a therapeutically effective amount of
(i) compound 1:

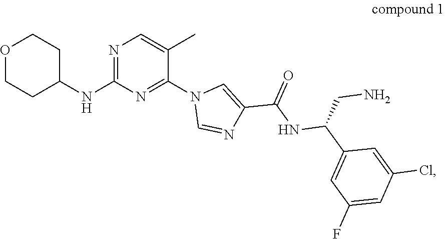 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

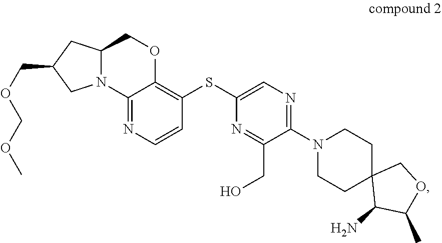 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) osimertinib.
(iii) an EGFR inhibitor.

Disclosed herein is a method of treating cancer in a subject in need thereof, the method comprising: administering to the subject in need thereof a therapeutically effective amount of
(i) compound 1:

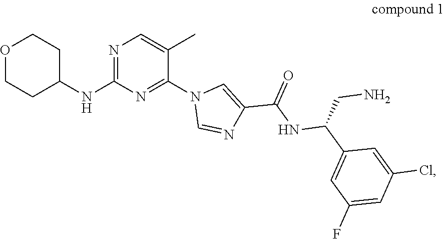 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

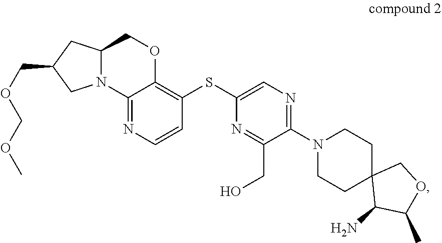 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) cetuximab.

Disclosed herein is a method of treating cancer in a subject in need thereof, the method comprising: administering to the subject in need thereof a therapeutically effective amount of
(i) compound 1:

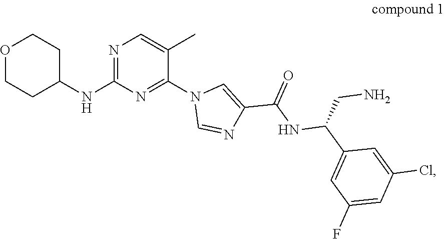 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

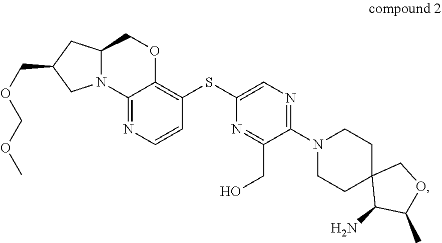 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof;
(iii) a BRAF inhibitor; and
(iv) an EGFR inhibitor.

Disclosed herein is a method of treating cancer in a subject in need thereof, the method comprising: administering to the subject in need thereof a therapeutically effective amount of
(i) compound 1:

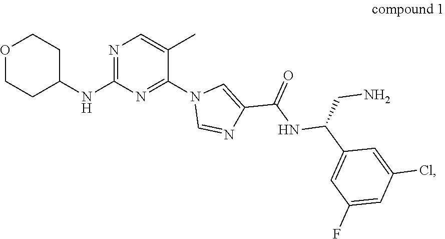 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

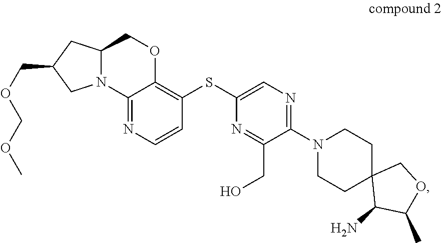 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof;
(iii) encorafenib; and
(iv) osimertinib.

Disclosed herein is a method of treating cancer in a subject in need thereof, the method comprising: administering to the subject in need thereof a therapeutically effective amount of
(i) compound 1:

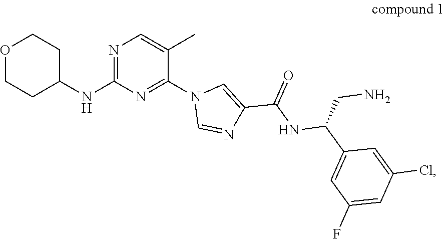 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

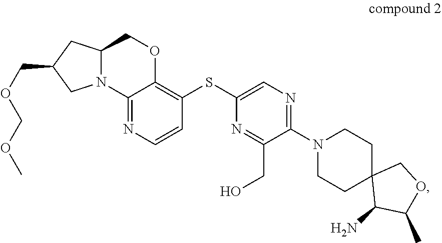 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof;
(iii) encorafenib; and
(iv) cetuximab.

Disclosed herein is a method of treating cancer in a subject in need thereof, the method comprising: administering to the subject in need thereof a therapeutically effective amount of
(i) compound 1:

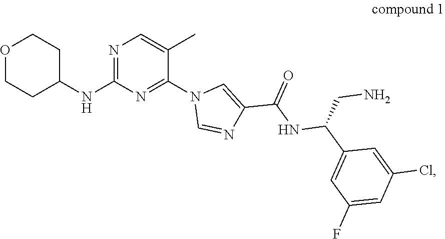 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

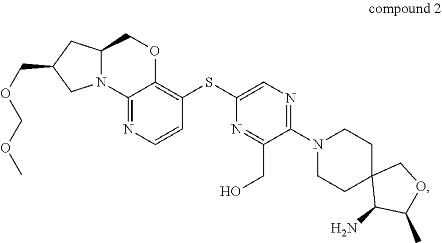 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) a FLT3 inhibitor.

Disclosed herein is a method of treating cancer in a subject in need thereof, the method comprising: administering to the subject in need thereof a therapeutically effective amount of
(i) compound 1:

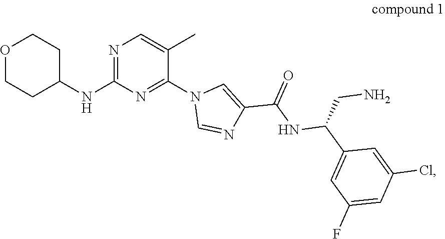 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

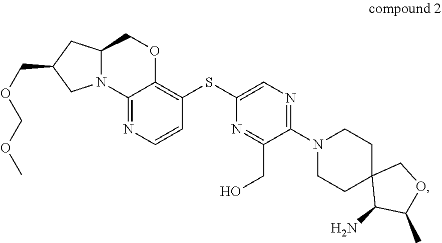 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) gilteritinib.

Disclosed herein is a method of treating cancer in a subject in need thereof, the method comprising: administering to the subject in need thereof a therapeutically effective amount of
(i) compound 1:

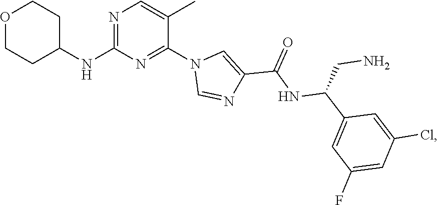 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

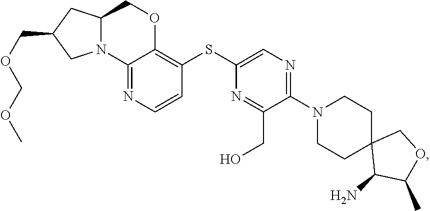 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) KRAS G12C inhibitor.

Disclosed herein is a method of treating cancer in a subject in need thereof, the method comprising: administering to the subject in need thereof a therapeutically effective amount of
(i) compound 1:

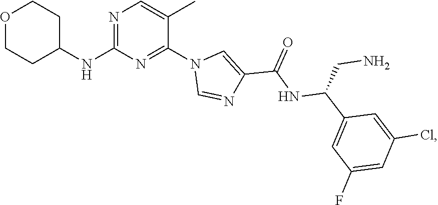 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

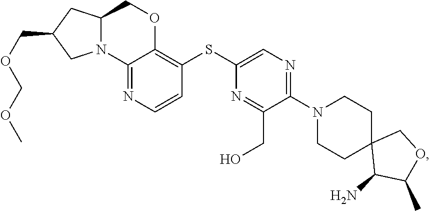 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) sotorasib.

Disclosed herein is a method of treating cancer in a subject in need thereof, the method comprising: administering to the subject in need thereof a therapeutically effective amount of
(i) compound 1:

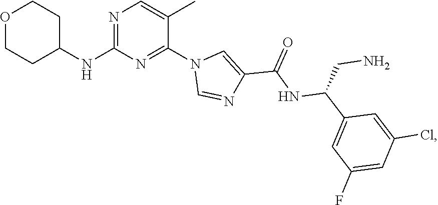 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

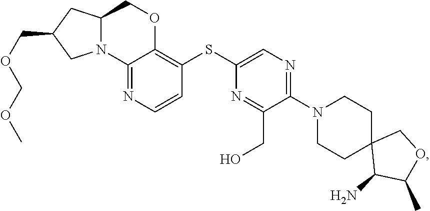 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) adagrasib.

Disclosed herein is a method of treating cancer in a subject in need thereof, the method comprising: administering to the subject in need thereof a therapeutically effective amount of
(i) compound 1:

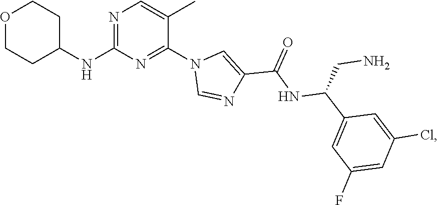 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

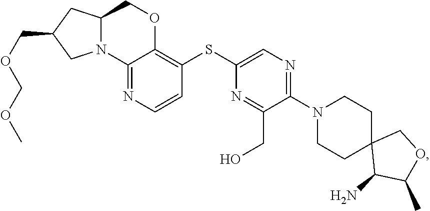 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) CDK4/6 inhibitor.

Disclosed herein is a method of treating cancer in a subject in need thereof, the method comprising: administering to the subject in need thereof a therapeutically effective amount of
(i) compound 1:

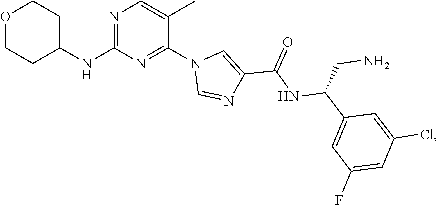 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

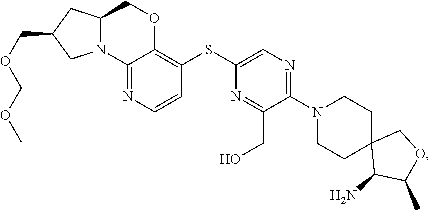 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) palbociclib.

### Further Combination

In some embodiments, the method comprises administering an additional MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In some embodiments, the additional MAPK pathway inhibitor inhibits a protein in the MAPK pathway. In some embodiments, the additional MAPK pathway inhibitor inhibits a protein outside the MAPK pathway. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. Exemplary MAPK pathway inhibitors include, without limitation, adagrasib, afatinib, ASTX029, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, panitumumab, selumetinib, sotorasib, trametinib, ulixertinib, vandetanib, and vemurafenib.

In some embodiment the additional MAPK pathway inhibitor is adagrasib. In some embodiment the additional MAPK pathway inhibitor is afatinib. In some embodiment the additional MAPK pathway inhibitors is binimetinib. In some embodiment the additional MAPK pathway inhibitor is cetuximab. In some embodiment the additional MAPK pathway inhibitor is cobimetinib. In some embodiment the additional MAPK pathway inhibitor is dabrafenib. In some embodiment the additional MAPK pathway inhibitor is dacomitinib. In some embodiment the additional MAPK pathway inhibitor is encorafenib. In some embodiment the additional MAPK pathway inhibitor is erlotinib. In some embodiment the additional MAPK pathway inhibitor is gefitinib. In some embodiment the additional MAPK pathway inhibitor is gilteritinib. In some embodiment the additional MAPK pathway inhibitor is lapatinib. In some embodiment the additional MAPK pathway inhibitor is LTT462. In some embodiment the additional MAPK pathway inhibitor is LY3214996. In some embodiment the additional MAPK pathway inhibitor is necitumumab. In some embodiment the additional MAPK pathway inhibitor is neratinib. In some embodiment the additional MAPK pathway inhibitor is nimotuzumab. In some embodiment the additional MAPK pathway inhibitor is osimertinib. In some embodiment the additional MAPK pathway inhibitor is palbociclib. In some embodiment the additional MAPK pathway inhibitor is panitumumab. In some embodiment the additional MAPK pathway inhibitor is selumetinib. In some embodiment the additional MAPK pathway inhibitor is sotorasib. In some embodiment the additional MAPK pathway inhibitor is trametinib. In some embodiment the additional MAPK pathway inhibitor is ulixertinib. In some embodiment the additional MAPK pathway inhibitor is vandetanib.

### Cancers

Disclosed herein are methods of treating cancer using a combination disclosed herein.

“Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In some embodiments, the cancer has a class 1 B-Raf mutation.

In some embodiments, the cancer harbors at least one of a EGFR, KRAS, BRAF (e.g., BRAF class III) and/or NF1 (e.g., loss of function) mutations.

In some embodiments, the mutant B-Raf comprises a V600 mutation. In some embodiments, the mutant of B-Raf comprises the mutation V600E. In some embodiments, the mutation is V600K. In some embodiments, the mutation is V600D. In some embodiments, the mutation is V600L. In some embodiments, the mutation is V600R. In some embodiments, the cancer is a BRAF V600E or V600K mutant tumor.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12R KRAS mutation. In some embodiments, the cancer has a G12S KRAS mutation. In some embodiments, the cancer has a G12V KRAS mutation. In some embodiments, the cancer has G12W KRAS mutation. In some embodiments, the cancer has a G13D KRAS mutation. In some embodiments, the cancer has a H95D KRAS mutation. In some embodiments, the cancer has a H95Q KRAS mutation. In some embodiments, the cancer has a H95R KRAS mutation. In some embodiments, the cancer has a Q16H KRAS mutation. In some embodiments, the cancer has a Q61H KRAS mutation. In some embodiments, the cancer has a Q16K KRAS mutation. In some embodiments, the cancer has a Q61R NRAS mutation. In some embodiments, the cancer has a R68S KRAS mutation.

In some embodiments, the cancer is a MAPKm/MAPKi-naïve pancreatic cancer. In some embodiments, the pancreatic cancer is pancreatic ductal adenocarcinoma (PDAC).

In some embodiments, the cancer comprises one or more EGFR mutation selected from the group consisting of EGFR gene copy gain, EGFR gene amplification, chromosome 7 polysomy, L858R, exon 19 deletions/insertions, L718Q, L861Q, G719C, G719S, G724S, G719A, V765A, T783A, exon 20 insertions, EGFR splice variants (Viii, Vvi, and Vii), A289D, A289T, A289V, G598A, G598V, T790M, S768I, C797X, and C797S. In some embodiments, the cancer comprises one or more EGFR mutation selected from the group consisting of L858R, exon 19 deletion, and T790M.

In some embodiments, the cancer is the cancer is a liquid tumor. In some embodiments, the cancer is the liquid tumor is leukemia. In some embodiments, the cancer is the leukemia is acute myeloid leukemia (AML). In some embodiments, the cancer is the AML is relapsed and/or refractory AML. In some embodiments, the cancer is the AML is a FLT3 mutant AML.

In some embodiments, the cancer is a solid tumor. In some embodiments, the solid tumor is an advanced or a metastatic solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma.

In some embodiments, the cancer is a gastrointestinal cancer. In some embodiments, the gastrointestinal is anal cancer, bile duct cancer, colon cancer, rectal cancer, esophageal cancer, gallbladder cancer, liver cancer, pancreatic cancer, small intestine cancer, or stomach cancer (gastric cancer).

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC. In some embodiments, the EGFR mutation is an acquired EGFR mutation. In some embodiments, the acquired EGFR mutation is C797X. In some embodiments, the acquired EGFR mutation is L718Q. In some embodiments, the acquired EGFR mutation is EGFR gene amplification. In some embodiments, the acquired EGFR mutation is G724S. In some embodiments, the acquired EGFR mutation is S768I.

In some embodiments, the NSCLC is a NRAS Q61R mutant NSCLC. In some embodiments, the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC. In some embodiments, the cancer is a KRAS-treated G12R NSCLC. In some embodiments, the cancer is a KRAS-treated G12W NSCLC. In some embodiments, the cancer is a KRAS-treated H95D NSCLC. In some embodiments, the cancer is a KRAS-treated H95Q NSCLC. In some embodiments, the cancer is a KRAS-treated H95R NSCLC. In some embodiments, the cancer is a KRAS-treated G12D NSCLC. In some embodiments, the cancer is a KRAS-treated R68S NSCLC.

In some embodiments, the cancer is pancreatic cancer. In some embodiments, the cancer is a MAPKm/MAPKi-naïve pancreatic cancer. In some embodiments, the pancreatic cancer is pancreatic ductal adenocarcinoma (PDAC).

In some embodiments, the cancer is melanoma. In some embodiments, the melanoma is a BRAF V600E or V600K mutant tumor. In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC.

In some embodiments, the CRC is a KRAS G12C mutant CRC. In some embodiments, the CRC is a KRAS G12D mutant CRC. In some embodiments, the CRC is a KRAS G12R mutant CRC. In some embodiments, the CRC is a KRAS G12S mutant CRC. In some embodiments, the CRC is a KRAS G12V mutant CRC. In some embodiments, the CRC is a KRAS G12W mutant CRC. In some embodiments, the CRC is a KRAS G13D mutant CRC. In some embodiments, the CRC has a H95D KRAS mutation. In some embodiments, the CRC has a H95Q KRAS mutation. In some embodiments, the CRC has a H95R KRAS mutation. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC. In some embodiments, the CRC has a R68S KRAS mutation.

In some embodiments, the cancer is esophageal cancer.

In some embodiments, the cancer has one or more acquired mutations. In some embodiments, the acquired mutation results from a first-line treatment. In some embodiments, the first-line treatment is an EGFR inhibitor. In some embodiments, the EGFR inhibitor is osimertinib. In some embodiments, the first-line treatment is a KRAS inhibitor. In some embodiments, the KRAS inhibitor is a KRAS G12C inhibitor. In some embodiments, the KRAS G12C inhibitor is adagrasib. In some embodiments, the KRAS G12C inhibitor is sotorasib. In some embodiments, the cancer is a solid tumor cancer. In some embodiments, the cancer is NSCLC.

In some embodiments, the acquired mutation is an acquired EGFR mutation. In some embodiments, the acquired EGFR mutation is C797X. In some embodiments, the acquired EGFR mutation is L718Q. In some embodiments, the acquired EGFR mutation is EGFR amplification. In some embodiments, the acquired EGFR mutation is G724S. In some embodiments, the acquired mutation is S768I.

In some embodiments, the acquired mutation is an acquired amplification mutation. In some embodiments, the acquired mutation is a MET gene amplification. In some embodiments, the acquired mutation is HER2 gene amplification.

In some embodiments, the acquired mutation is an acquired oncogenic fusion. In some embodiments, the acquired oncogenic fusion is SPTBN1-ALK. In some embodiments, the acquired oncogenic fusion is RET fusion. In some embodiments, the acquired oncogenic fusion is BRAF fusion.

In some embodiments, the acquired mutation is an acquired MAPK-PI3K mutation. In some embodiments, the acquired MAPK-PI3K mutation is BRAF-V600E. In some embodiments, the acquired MAPK-PI3K mutation is PI3KCA. In some embodiments, the acquired MAPK-PI3K mutation is KRAS. In some embodiments, the acquired MAPK-PI3K mutation is HER2.

In some embodiments, the acquired mutation is an acquired KRAS mutation. In some embodiments, the acquired mutation is KRAS G12C. In some embodiments, the acquired mutation is KRAS G12D. In some embodiments, the acquired mutation is KRAS G12R. In some embodiments, the acquired mutation is KRAS G12V. In some embodiments, the acquired mutation is KRAS G12W. In some embodiments, the acquired mutation is KRAS G13D. In some embodiments, the acquired mutation is KRAS H95D. In some embodiments, the acquired mutation is KRAS H95D. In some embodiments, the acquired mutation is KRAS H95Q. In some embodiments, the acquired mutation is KRAS H95R. In some embodiments, the acquired mutation is KRAS Q61H. In some embodiments, the acquired mutation is KRAS R68S.

In some embodiments, the acquired mutation is an acquired MAPK pathway mutation. In some embodiments, the acquired MAPK pathway mutation is MAP2K1 K57N. In some embodiments, the acquired MAPK pathway mutation is MAP2K1 K57T. In some embodiments, the acquired MAPK pathway mutation is CCDC6-RET. In some embodiments, the acquired MAPK pathway mutation is RITI P128L. In some embodiments, the acquired MAPK pathway mutation is PTEN G209V. In some embodiments, the acquired MAPK pathway mutation is BRAF V600E. In some embodiments, the acquired MAPK pathway mutation is MAP2K1 199_K04del. In some embodiments, the acquired MAPK pathway mutation is MAP2K1 K57N. In some embodiments, the acquired MAPK pathway mutation is EML4-ALK. In some embodiments, the acquired MAPK pathway mutation is EGFR A289A. In some embodiments, the acquired MAPK pathway mutation is FGFR3-TACC3. In some embodiments, the acquired MAPK pathway mutation is AKAP9-BRAF. In some embodiments, the acquired MAPK pathway mutation is RAF1-CCDCl76. In some embodiments, the acquired MAPK pathway mutation is RAF1-TRAK1. In some embodiments, the acquired MAPK pathway mutation is NRAS Q61K. In some embodiments, the acquired MAPK pathway mutation is MAP2K1 E102_1103DEL. In some embodiments, the acquired MAPK pathway mutation is NRF1-BRAF.

In some embodiments, the acquired mutation is a KRAS G12C reactivation mutation. In some embodiments, the KRAS G12C reactivation mutation is a RKRAS G12C gene amplification. In some embodiments, the KRAS G12C reactivation mutation is a NF1 R22637 (LoF).

In some embodiments, the acquired mutation is a non-G12C activation KRAS mutation. In some embodiments, the non-G12C activation KRAS mutation is KRAS G12D. In some embodiments, the non-G12C activation KRAS mutation is KRAS G12R. In some embodiments, the non-G12C activation KRAS mutation is KRAS G12V. In some embodiments, the non-G12C activation KRAS mutation is KRAS G12W. In some embodiments, the non-G12C activation KRAS mutation is KRAS G13D. In some embodiments, the non-G12C activation KRAS mutation is KRAS Q61H. In some embodiments, the non-G12C activation KRAS mutation is KRAS Q61K.

In some embodiments, the acquired mutation is a sterically hindering KRAS G12C mutation. In some embodiments, the sterically hindering KRAS G12C mutation is KRAS R68S. In some embodiments, the sterically hindering KRAS G12C mutation is KRAS H95D. In some embodiments, the sterically hindering KRAS G12C mutation is KRAS H95Q. In some embodiments, the sterically hindering KRAS G12C mutation is KRAS H95R. In some embodiments, the sterically hindering KRAS G12C mutation is KRAS Y96C.

In some embodiments, the acquired mutation is an RTK activation mutation. In some embodiments, the RTK activation mutation is EGFR A289V. In some embodiments, the RTK activation mutation is RET M918T. In some embodiments, the RTK activation mutation is MET gene amplification. In some embodiments, the RTK activation mutation is EML-ALK. In some embodiments, the RTK activation mutation is CCDC6-RET. In some embodiments, the RTK activation mutation is FGFR3-TACC3.

In some embodiments, the acquired mutation is a downstream RAS/MAPK activation mutation. In some embodiments, the downstream RAS/MAPK activation mutation is BRAF V600E. In some embodiments, the downstream RAS/MAPK activation mutation is MAP2K I99_K104del. In some embodiments, the downstream RAS/MAPK activation mutation is MAP2K1 I99_K104del. In some embodiments, the downstream RAS/MAPK activation mutation is MAP2K1 E102_I103del. In some embodiments, the downstream RAS/MAPK activation mutation is RAF fusion.

In some embodiments, the acquired mutation is a parallel pathway activation mutation. In some embodiments, the parallel pathway activation mutation is PIK3CA H1047R. In some embodiments, the parallel pathway activation mutation is PIK3R1 S361fs. In some embodiments, the parallel pathway activation mutation is PTEN N48K. In some embodiments, the parallel pathway activation mutation is PTEN G209V. In some embodiments, the parallel pathway activation mutation is RIT1 P128L.

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or a pharmaceutically acceptable salt thereof can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or a pharmaceutically acceptable salt thereof that corresponds to such an amount varies depending upon factors such as the molecular weight of a particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, the amount of compound 1, or a pharmaceutically acceptable salt thereof, as described herein is relative to the free-base equivalent of compound 1

In some embodiments, compound 1, or a pharmaceutically acceptable salt thereof, is administered orally.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 150 mg/day, about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 1, or a pharmaceutically acceptable salt thereof, is administered in an amount between about 25 mg to about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 200 twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is between about 25 mg and about 100 mg twice a day, once a week (BID-QW).

In some embodiments, compound 1, or a pharmaceutically acceptable salt thereof, is administered in an amount that is between about 25 mg and about 50 mg twice a day, once a week (BID-QW).

In some embodiments, compound 1, or a pharmaceutically acceptable salt thereof, is administered in an amount that is between about 50 mg to about 300 mg twice a day, once a week (BID-QW).

In some embodiments, compound 1, or a pharmaceutically acceptable salt thereof, is administered in an amount that is between about 50 mg and about 250 twice a day, once a week (BID-QW).

In some embodiments, compound 1, or a pharmaceutically acceptable salt thereof, is administered in an amount that is between about 50 mg and about 200 mg twice a day, once a week (BID-QW).

In some embodiments, compound 1, or a pharmaceutically acceptable salt thereof, is administered in an amount that is between about 50 mg and about 150 mg twice a day, once a week (BID-QW).

In some embodiments, compound 1, or a pharmaceutically acceptable salt thereof, is administered in an amount that is between about 50 mg and about 100 mg twice a day, once a week (BID-QW).

In some embodiments, compound 1, or a pharmaceutically acceptable salt thereof, is administered in an amount that is between about 100 mg and about 300 mg twice a day, once a week (BID-QW).

In some embodiments, compound 1, or a pharmaceutically acceptable salt thereof, is administered in an amount that is between about 100 mg and about 250 mg twice a day, once a week (BID-QW).

In some embodiments, compound 1, or a pharmaceutically acceptable salt thereof, is administered in an amount that is between about 100 mg and about 200 mg twice a day, once a week (BID-QW).

In some embodiments, compound 1, or a pharmaceutically acceptable salt thereof, is administered in an amount that is between about 100 mg and about 150 mg twice a day, once a week (BID-QW).

In some embodiments, compound 1, or a pharmaceutically acceptable salt thereof, is administered in an amount that is between about 150 mg and about 300 mg twice a day, once a week (BID-QW).

In some embodiments, compound 1, or a pharmaceutically acceptable salt thereof, is administered in an amount that is between about 150 mg and about 250 mg twice a day, once a week (BID-QW).

In some embodiments, compound 1, or a pharmaceutically acceptable salt thereof, is administered in an amount that is between about 150 mg and about 200 mg twice a day, once a week (BID-QW).

In some embodiments, compound 1, or a pharmaceutically acceptable salt thereof, is administered in an amount that is between about 175 mg and about 300 mg twice a day, once a week (BID-QW).

In some embodiments, compound 1, or a pharmaceutically acceptable salt thereof, is administered in an amount that is between about 175 mg and about 250 mg twice a day, once a week (BID-QW).

In some embodiments, compound 1, or a pharmaceutically acceptable salt thereof, is administered in an amount that is between about 175 mg and about 200 mg twice a day, once a week (BID-QW).

In some embodiments, compound 1, or a pharmaceutically acceptable salt thereof, is administered in an amount that is between about 200 mg and about 300 mg twice a day, once a week (BID-QW).

In some embodiments, compound 1, or a pharmaceutically acceptable salt thereof, is administered in an amount that is between about 200 mg and about 250 mg twice a day, once a week (BID-QW).

In some embodiments, compound 1, or a pharmaceutically acceptable salt thereof, is administered in an amount that is between about 225 mg and about 300 mg twice a day, once a week (BID-QW).

In some embodiments, compound 1, or a pharmaceutically acceptable salt thereof, is administered in an amount that is between about 225 mg and about 250 mg twice a day, once a week (BID-QW).

In some embodiments, compound 1, or a pharmaceutically acceptable salt thereof, is administered in an amount that is between about 25 mg and about 300 mg once a week (QW).

In some embodiments, compound 1, or a pharmaceutically acceptable salt thereof, is administered in an amount that is between about 50 mg and about 250 mg once a week (QW).

In some embodiments, compound 1, or a pharmaceutically acceptable salt thereof, is administered in an amount that is between about 100 mg and about 300 mg once a week (QW).

In some embodiments, compound 1, or a pharmaceutically acceptable salt thereof, is administered in an amount that is between about 100 mg and about 250 mg once a week (QW).

In some embodiments, compound 1, or a pharmaceutically acceptable salt thereof, is administered in an amount that is between about 150 mg and about 300 mg once a week (QW).

In some embodiments, compound 1, or a pharmaceutically acceptable salt thereof, is administered in an amount that is between about 150 mg and about 250 mg once a week (QW).

In some embodiments, compound 1, or a pharmaceutically acceptable salt thereof, is administered in an amount that is about 100 mg once a week (QW). In some embodiments, compound 1, or a pharmaceutically acceptable salt thereof, is administered in an amount that is about 150 mg once a week (QW). In some embodiments, compound 1, or a pharmaceutically acceptable salt thereof, is administered in an amount that is about 200 mg once a week (QW). In some embodiments, compound 1, or a pharmaceutically acceptable salt thereof, is administered in an amount that is about 250 mg once a week (QW).

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered in an amount that is about 250 mg once a day, once a week.

In some embodiments, compound 1, or a pharmaceutically acceptable salt thereof, is administered in an amount that is about 25 mg, 30 mg, 40 mg, 50 mg, about 60 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

In some embodiments, each of the above-recited amounts may be administered QD, QW, BID, BID-QD, or BID-QW.

In some embodiments, compound 2, or a pharmaceutically acceptable salt thereof, is administered orally.

In some embodiments, compound 2, or a pharmaceutically acceptable salt thereof, is administered in an amount that is between about 1 mg/day and about 500 mg/day.

In some embodiments, compound 2, or a pharmaceutically acceptable salt thereof, is administered in an amount that is between about 20 mg/day and about 400 mg/day.

In some embodiments, compound 2, or a pharmaceutically acceptable salt thereof, is administered in an amount that is between about 30 mg/day and about 300 mg/day.

In some embodiments, compound 2, or a pharmaceutically acceptable salt thereof, is administered in an amount that is about 25 mg/day, about 50 mg/day, about 75 mg/day, about 100 mg/day, about 150 mg/day, about 175 mg/day, about 200 mg/day, about 225 mg/day, about 250 mg/day, about 275 mg/day, about 300 mg/day, about 325 m/day, about 350 mg/day, or about 400 mg/day.

In some embodiments, compound 2, or a pharmaceutically acceptable salt thereof, is administered QD or BID for 2 weeks on and 1 week off (21 day schedule). In some embodiments, compound 2, or a pharmaceutically acceptable salt thereof, is administered QD or BID for 3 weeks on and 1 week off (28 day schedule). In some embodiments, compound 2, or a pharmaceutically acceptable salt thereof, is administered QD or BID three times a week (D1D3D5 TIW) e.g., Day 1, Day 3, and Day 5. In some embodiments, compound 2, or a pharmaceutically acceptable salt thereof, is administered twice a day/twice a week e.g., Day 1 and Day 2 (BID-D1D2-BlW).

In some embodiments, compound 2, or a pharmaceutically acceptable salt thereof, is administered once a day (QD) continuous dosing at a dose of 20 mg/day to 60 mg/day, 40 mg/day, or 60 mg/day. In some embodiments, compound 2, or a pharmaceutically acceptable salt thereof, is administered twice a day (BID) continuous dosing at a dose of 20 mg/day to 80 mg/day. In some embodiments, compound 2, or a pharmaceutically acceptable salt thereof, is administered twice a day (BID) continuous dosing at a dose of 10 mg/day to 100 mg/day.

In some embodiments, each of the above-recited amounts may be administered QD, QW, BID, BID-QD, or BID-QW.

In some embodiments, osimertinib is administered in an amount that is between about 50/day mg and about 300 mg/day. In some embodiments, osimertinib is administered in an amount that is about 20 mg/day. In some embodiments, osimertinib is administered in an amount that is about 40 mg/day. In some embodiments, osimertinib is administered in an amount that is about 60 mg/day. In some embodiments, osimertinib is administered in an amount that is about 80 mg/day. In some embodiments, osimertinib is administered in an amount that is about 100 mg/day. In some embodiments osimertinib is administered in an amount that is about 120 mg/day. In some embodiments, osimertinib is administered in an amount that is about 140 mg/day.

In some embodiments, cetuximab is administered at 400 mg/m2 over 120 minutes followed by 250 mg/m2 over 60 minutes once a week. In some embodiments, cetuximab is administered at 500 mg/m2 once every two weeks. In some embodiments, cetuximab is administered at 400 mg/m2 once every two weeks. In some embodiments, cetuximab is administered at 300 mg/m2 once every two weeks.

In some embodiments, encorafenib is administered in an amount that is between about 100 mg/day and about 500 mg/day. In some embodiments, encorafenib is administered in an amount that is about 450 mg/day. In some embodiments, encorafenib is administered in an amount that is about 300 mg/day. In some embodiments, encorafenib is administered in an amount that is about 225 mg/day. In some embodiments, encorafenib is administered in an amount that is about 150 mg/day.

In some embodiments, dabrafenib is administered in an amount that is between about 100 mg/day and about 500 mg/day. In some embodiments, dabrafenib is administered in an amount that is about 450 mg/day. In some embodiments, dabrafenib is administered in an amount that is about 300 mg/day. In some embodiments, dabrafenib is administered in an amount that is about 225 mg/day. In some embodiments, dabrafenib is administered in an amount that is about 150 mg/day. In some embodiments, dabrafenib is administered in an amount that is about 100 mg/day.

In some embodiments, gilteritinib is administered in an amount that is between about 100 mg/day and about 500 mg/day. In some embodiments, gilteritinib is administered in an amount that is about 450 mg/day. In some embodiments, gilteritinib is administered in an amount that is about 300 mg/day. In some embodiments, gilteritinib is administered in an amount that is about 225 mg/day. In some embodiments, gilteritinib is administered in an amount that is about 150 mg/day. In some embodiments, gilteritinib is administered in an amount that is about 120 mg/day. In some embodiments, gilteritinib is administered in an amount that is about 100 mg/day.

In some embodiments, sotorasib is administered in an amount that is between about 500 mg/day and about 1500 mg/day. In some embodiments, sotorasib is administered in an amount that is about 1000 mg/day. In some embodiments, sotorasib is administered in an amount that is about 960 mg/day. In some embodiments, sotorasib is administered in an amount that is about 900 mg/day. In some embodiments, sotorasib is administered in an amount that is about 800 mg/day. In some embodiments, sotorasib is administered in an amount that is about 700 mg/day. In some embodiments, sotorasib is administered in an amount that is about 600 mg/day. In some embodiments, sotorasib is administered in an amount that is about 500 mg/day.

In some embodiments, adagrasib is administered in an amount that is between about 500 mg/day and about 1500 mg/day. In some embodiments, adagrasib is administered in an amount that is about 1200 mg/day. In some embodiments, adagrasib is administered in an amount that is about 1000 mg/day. In some embodiments, adagrasib is administered in an amount that is about 800 mg/day. In some embodiments, adagrasib is administered in an amount that is about 600 mg/day. In some embodiments, adagrasib is administered in an amount that is about 400 mg/day.

In some embodiments, palbociclib is administered in an amount that is between about 50 mg/day and about 500 mg/day. In some embodiments, palbociclib is administered in an amount that is about 150 mg/day. In some embodiments, palbociclib is administered in an amount that is about 125 mg/day. In some embodiments, palbociclib is administered in an amount that is about 100 mg/day. In some embodiments, palbociclib is administered in an amount that is about 75 mg/day. In some embodiments, palbociclib is administered in an amount that is about 50 mg/day.

In some embodiments, palbociclib is administered in an amount that is between about 50 mg once a week and about 650 mg once a week. In some embodiments, palbociclib is administered in an amount that is between about 600 mg once a week. In some embodiments, palbociclib is administered in an amount that is between about 500 mg once a week. In some embodiments, palbociclib is administered in an amount that is between about 400 mg once a week. In some embodiments, palbociclib is administered in an amount that is between about 300 mg once a week. In some embodiments, palbociclib is administered in an amount that is between about 200 mg once a week.

### Administration

Administration of compound 1, or a pharmaceutically acceptable salt thereof, and combination partners described herein are at a dosage described herein or at other dose levels and compositions determined and contemplated by a medical practitioner. In certain embodiments, compound 1, or a pharmaceutically acceptable salt thereof, is administered for prophylactic and/or therapeutic treatments. In certain therapeutic applications, compound 1, or a pharmaceutically acceptable salt thereof, and combination partners described herein, are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, compound 1, or a pharmaceutically acceptable salt thereof, and combination partners described herein, are administered once a day. In some embodiments, compound 1, or a pharmaceutically acceptable salt thereof, and combination partners described herein are administered twice a day. In some embodiments, compound 1, or a pharmaceutically acceptable salt thereof, and combination partners described herein are administered three times a day.

In some embodiments, compound 2, or a pharmaceutically acceptable salt thereof, is administered once daily. In some embodiments, compound 2, or a pharmaceutically acceptable salt thereof, is administered twice daily. In some embodiments, compound 2, or a pharmaceutically acceptable salt thereof, is administered three times daily.

In some embodiments, compound 1, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 1, or a pharmaceutically acceptable salt thereof, is administered to a subject who is in a fasted state for at least 8 hours. In other embodiments, compound 1, or a pharmaceutically acceptable salt thereof, and combination partners described herein, are administered to a subject who is in a fasted state for at least 10 hours. In yet other embodiments, compound 1, or a pharmaceutically acceptable salt thereof, and combination partners described herein, are administered to a subject who is in a fasted state for at least 12 hours. In other embodiments, compound 1, or a pharmaceutically acceptable salt thereof, and combination partners described herein, are administered to a subject who has fasted overnight.

In other embodiments, compound 1, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, compound 1, or a pharmaceutically acceptable salt thereof, is administered to a subject in a fed state 30 minutes post-meal. In other instances, compound 1, or a pharmaceutically acceptable salt thereof, and combination partners described herein, are administered to a subject in a fed state 1 hour post-meal. In yet further embodiments, compound 1, or a pharmaceutically acceptable salt thereof, is administered to a subject with food.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

In some embodiments of a method of treating cancer, compound 1, or a pharmaceutically acceptable salt thereof, and combination partners described herein are administered in 28-day cycles. In some embodiments of a method of treating cancer, compound 1, or a pharmaceutically acceptable salt thereof, and combination partners described herein, are administered for multiple 28-day cycles. In some embodiments of a method of treating cancer, compound 1, or a pharmaceutically acceptable salt thereof, and combination partners described herein, are administered for at least one 28-day cycle. In some embodiments of a method of treating cancer, compound 1, or a pharmaceutically acceptable salt thereof, and combination partners described herein, are administered for at least two 28-day cycles. In some embodiments of a method of treating cancer, compound 1, or a pharmaceutically acceptable salt thereof, and combination partners described herein, are administered for at least three 28-day cycles. In some embodiments of a method of treating cancer, compound 1, or a pharmaceutically acceptable salt thereof, and combination partners described herein, are administered for at least four 28-day cycles. In some embodiments of a method of treating cancer, compound 1, or a pharmaceutically acceptable salt thereof, and combination partners described herein, are administered for at least five 28-day cycles. In some embodiments of a method of treating cancer, compound 1, or a pharmaceutically acceptable salt thereof, and combination partners described herein, are administered for at least six 28-day cycles.

In some embodiments of a method of treating cancer, compound 1, or a pharmaceutically acceptable salt thereof, is administered on days 1-7 of each 28-day cycle. In some embodiments of a method of treating cancer, compound 1, or a pharmaceutically acceptable salt thereof, is administered on days 1-14 of each 28-day cycle. In some embodiments of a method of treating cancer, compound 1, or a pharmaceutically acceptable salt thereof, is administered on days 1-21 of each 28-day cycle. In some embodiments of a method of treating cancer, compound 1, or a pharmaceutically acceptable salt thereof, is administered on days 1-28 of each 28-day cycle.

In some embodiments of a method of treating cancer, compound 1, or a pharmaceutically acceptable salt thereof, is administered twice a day on day 1 of a 28-day cycle. In some embodiments of a method of treating cancer, compound 1, or a pharmaceutically acceptable salt thereof, is administered twice a day on day 8 of a 28-day cycle. In some embodiments of a method of treating cancer, compound 1, or a pharmaceutically acceptable salt thereof, is administered twice a day on day 15 of a 28-day cycle. In some embodiments of a method of treating cancer, compound 1, or a pharmaceutically acceptable salt thereof, is administered twice a day on day 22 of a 28-day cycle. In some embodiments of a method of treating cancer, compound 1, or a pharmaceutically acceptable salt thereof, is not administered twice a day on day 22 of a 28-day cycle.

In some embodiments of a method of treating cancer, compound 1, or a pharmaceutically acceptable salt thereof, is administered twice a day on day 1, day 8, and day 15 of a 28-day cycle.

In some embodiments of a method of treating cancer, compound 1, or a pharmaceutically acceptable salt thereof, is not administered on days 2-7, days 9-14, days 16-21, days 23-28 of a 28-day cycle.

In some embodiments of a method of treating cancer, compound 1, or a pharmaceutically acceptable salt thereof, and combination partners described herein are administered in 35-day cycles. In some embodiments of a method of treating cancer, compound 1, or a pharmaceutically acceptable salt thereof, and combination partners described herein are administered for multiple 35-day cycles. In some embodiments of a method of treating cancer, compound 1, or a pharmaceutically acceptable salt thereof, and combination partners described herein are administered for at least one 35-day cycle. In some embodiments of a method of treating cancer, compound 1, or a pharmaceutically acceptable salt thereof, and combination partners described herein are administered for at least two 35-day cycle. In some embodiments of a method of treating cancer, compound 1, or a pharmaceutically acceptable salt thereof, and combination partners described herein are administered for at least three 35-day cycle. In some embodiments of a method of treating cancer, compound 1, or a pharmaceutically acceptable salt thereof, and combination partners described herein are administered for at least four 35-day cycle. In some embodiments of a method of treating cancer, compound 1, or a pharmaceutically acceptable salt thereof, and combination partners described herein are administered for at least five 35-day cycle. In some embodiments of a method of treating cancer, compound 1, or a pharmaceutically acceptable salt thereof, and combination partners described herein are administered for at least six 35-day cycle.

In some embodiments of a method of treating cancer, compound 1, or a pharmaceutically acceptable salt thereof, is administered on days 1-7 of each 35-day cycle. In some embodiments of a method of treating cancer, compound 1, or a pharmaceutically acceptable salt thereof, is administered on days 1-14 of each 35-day cycle. In some embodiments of a method of treating cancer, compound 1, or a pharmaceutically acceptable salt thereof, is administered on days 1-21 of each 35-day cycle. In some embodiments of a method of treating cancer, compound 1, or a pharmaceutically acceptable salt thereof, is administered on days 1-28 of each 35-day cycle. In some embodiments of a method of treating cancer, compound 1, or a pharmaceutically acceptable salt thereof, is administered on days 1-35 of each 35-day cycle.

In some embodiments of a method of treating cancer, compound 1, or a pharmaceutically acceptable salt thereof, is administered twice a day on day 1 of a 35-day cycle. In some embodiments of a method of treating cancer, compound 1, or a pharmaceutically acceptable salt thereof, is administered twice a day on day 8 of a 35-day cycle. In some embodiments of a method of treating cancer, compound 1, or a pharmaceutically acceptable salt thereof, is administered twice a day on day 15 of a 35-day cycle. In some embodiments of a method of treating cancer, compound 1, or a pharmaceutically acceptable salt thereof, is administered twice a day on day 22 of a 35-day cycle. In some embodiments of a method of treating cancer, compound 1, or a pharmaceutically acceptable salt thereof, is administered twice a day on day 29 of a 35-day cycle. In some embodiments of a method of treating cancer, compound 1, or a pharmaceutically acceptable salt thereof, is not administered twice a day on day 29 of a 35-day cycle.

In some embodiments of a method of treating a cancer, compound 1, or a pharmaceutically acceptable salt thereof, is administered twice a day on day 1, day 8, day 15, and day 22 of a 35-day cycle.

In some embodiments of a method of treating cancer, compound 1, or a pharmaceutically acceptable salt thereof, is not administered on days 2-7, days 9-14, days 16-21, days 23-28, and days 30-35 of a 28-day cycle.

### EXAMPLES

### Example 1: In-Vivo Assay

The vehicle/control article, 0.5% Methyl Cellulose & 0.1% Tween 80 or 100 mM acetic acid in deionized water with pH adjustment to 4.8-5.0, was prepared and stored under ambient conditions throughout the study period.

### Formulation of Test Article

The test article, compound 1, was freshly prepared in vehicle of 0.5% Methyl Cellulose & 0.1% Tween 80 weekly and stored under ambient conditions. The combination agent, compound 2, was freshly prepared in vehicle of 100 mM acetic buffer weekly and stored under ambient conditions.

### Animals

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign and WuXi AppTec. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign and WuXi AppTec adhered to the study protocols reviewed and approved by IACUC and applicable standard operating procedures (SOPs).

### Preparation of Xenograft Model

NCI-H441 is a human NSCLC cell line that harbors a KRAS^{G12V} mutation. The NCI-H441 cell line was purchased from the American Type Culture Collection (ATCC® HTB-174™). NCI-H441 cells were cultured in medium containing RPMI-1640 plus 10% Fetal Bovine Serum (FBS), at 37° C. in an atmosphere of 5% CO₂ in air. NCI-H441 cells in 200 μL cell suspensions containing 5×10⁶ cells mixed with 50% Matrigel were implanted into mice subcutaneously. When tumor volumes reached a mean of 200 mm³, tumor-bearing mice were randomized into different groups with 8 mice in each group and treatment started on the day of randomization.

NCI-H2009 is a human NSCLC cell line that harbored a KRAS^{G12A} mutation. The NCI-H2009 cell line was purchased from the American Type Culture Collection (ATCC® CRL-5911). NCI-H2009 cells were cultured in medium containing RPMI-1640 plus 10% Fetal Bovine Serum (FBS) and supplemented with Glutamax and pyruvate at 37° C. in an atmosphere of 5% CO₂ in air. NCI-H2009 cells in 100 μL cell suspensions containing 5×10⁶ cells mixed with 50% Matrigel were implanted into mice subcutaneously. When tumor volumes reached a mean of 200 mm³, tumor-bearing mice were randomized into different groups with 8 mice in each group and treatment started on the day of randomization.

### Treatment

Mice were dosed by oral administration of vehicle control solution, compound 1, or compound 2 in monotherapy treatment groups. Mice were dosed by oral administration of combinations, including compound 1 with compound 2. The dosing volume was 5 mL/kg for each compound and interval of BID regimen was 8 hours. In the combination of compound 1 with compound 2, compound 1 was dosed at one-hour post compound 2 QD or first BID dose. In addition to regular food and water supply, DietGel (ClearH2O, US) was added in cages where at least two mice showed>10% BWL. The study was terminated at the end of 4-week treatment or when tumor volume in vehicle control group reached 2,000 mm³.

### Results

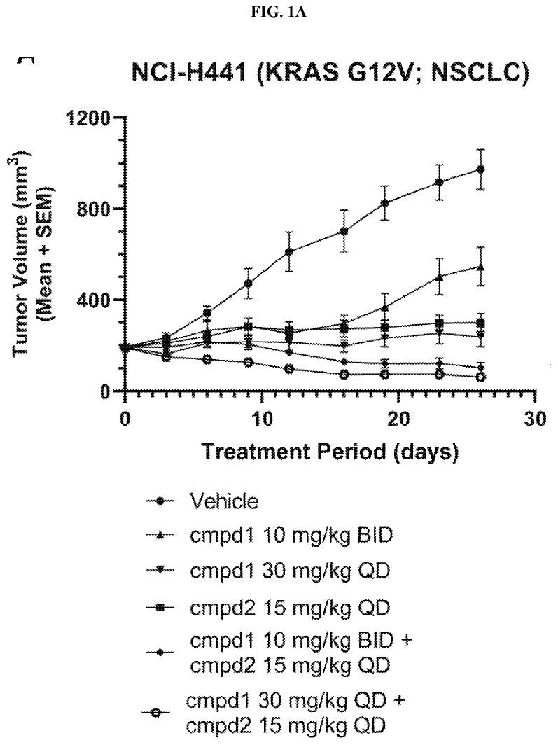

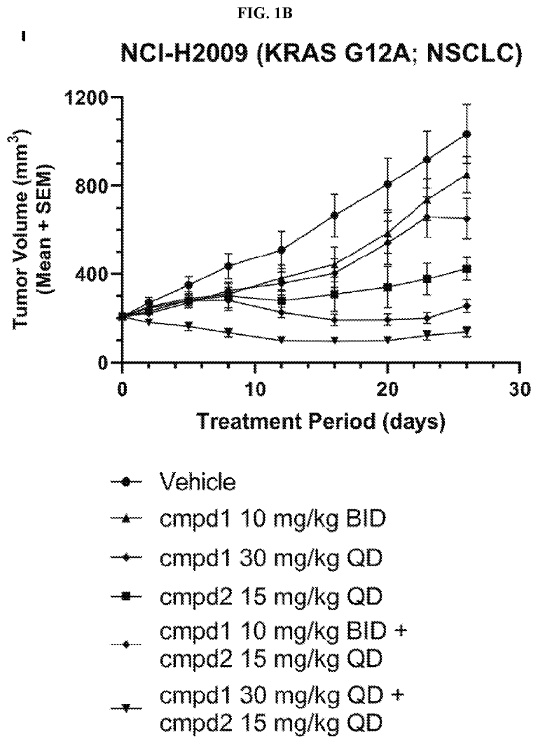

In the KRAS G12V NSCLC CDX model NCI-H441, as illustrated in FIG. 1A, the combination of compound 1 at 30 mg/kg QD and compound 2 at 15 mg/kg QD achieved a statistically significant TGI of 113% (p-value<0.001), demonstrating statistically significant benefit relative to the respective monotherapy doses of both compound 1 at 30 mg/kg QD and compound 2 at 15 mg/kg QD (p-value<0.01). Compound 1 as a monotherapy at 30 mg/kg BID and 30 mg/kg QD doses achieved statistically significant TGI of 115% (p-value<0.001) and 94% (p-value<0.001), respectively. Compound 2 as a monotherapy at 30 mg/kg QD and 15 mg/kg QD doses achieved statistically significant TGI of 101% (p-value<0.001) and 87% (p-value<0.001), respectively. FIG. 1A shows the tumor growth curves for this study.

In the KRAS G12A NSCLC CDX model NCI-H2009, as illustrated in FIG. 1B, the combination of compound 1 at 30 mg/kg QD and compound 2 at 15 mg/kg QD achieved statistically significant TGI of 107% (p-value<0.001). The combination of compound 1 and compound 2 achieved a statistically significant combination benefit relative to the respective monotherapy doses of both compound 1 at 30 mg/kg QD and compound 2 at 15 mg/kg QD (p-value<0.01). The combination also showed statistically significant superior TGI relative to compound 1 monotherapy at 30 mg/kg BID (p-value<0.05) and compound 2 monotherapy at 30 mg/kg QD (p-value<0.01). These doses represent the maximum monotherapy nonclinical efficacious doses for compound 1 and compound 2. Compound 1 as a monotherapy at 30 mg/kg BID achieved statistically significant TGI of 93%. Compound 2 as a monotherapy at 30 mg/kg QD and 15 mg/kg QD doses achieved statistically significant TGI of 90% (p-value<0.001) and 73% (p-value<0.001), respectively. FIG. 1B shows the tumor growth curves for this study.

Compound 1 and compound 2 demonstrated combination benefit in vivo on mutant RAS CDX models.

### Example 2: In Vitro Studies of Compound 1 Alone, Compound 2 Alone, and Compound 1+Compound 2 Combination in NSCLC Cell Lines

Cells (8000 cells per well) were plated onto 6-well plates in 2 ml cell culture medium. The cells were incubated overnight and treated with above indicated concentrations of Compound 1 and Compound 2. After 7 days incubation, the medium was replaced with fresh medium, cells were treated again with same concentration of Compound 1 and Compound 2 and incubated for additional 7 days. After 14 days of total incubation, the cells were washed with PBS twice and fixed 30 mins with 4% formaldehyde. The cells were washed twice with PBS and incubated with 0.1% crystal violet for 60 minutes. After the crystal violet staining, the cells were washed five times with water and dried at room temperature. After the plates were dried, the crystal staining was destained with 1 ml of 10% acetic acid and absorbance was measured at 560 nM.

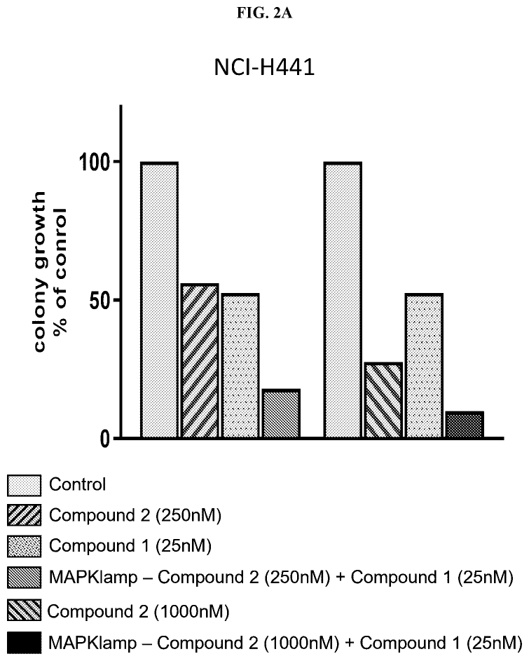

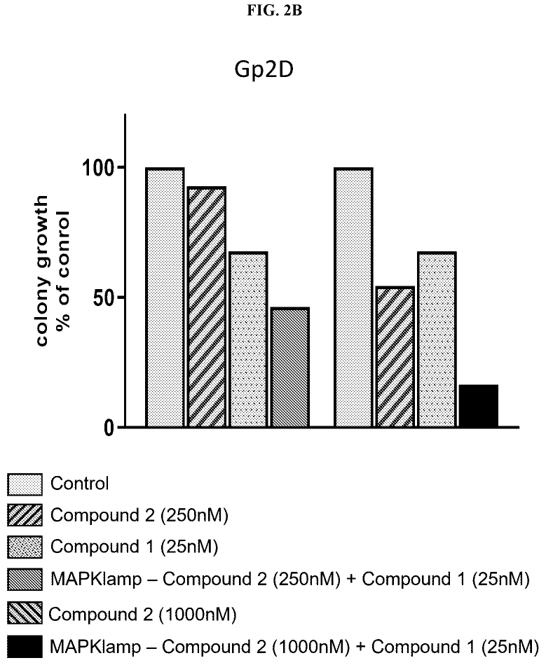

As illustrated by FIG. 2A, Compound 1 and Compound 2 demonstrated a combination benefit in the KRAS G12V mutant NSCLC Cell line, NCI-H441. As illustrated by FIG. 2B, Compound 1 and Compound 2 demonstrated a combination benefit in the KRAS G12D mutant NSCLC cell line, Gp2D.

### Example 3: In Vitro Studies of Compound 1 Alone, Compound 2 Alone, and Compound 1+Compound 2 Combination in Mutated Pancreatic and Pancreatic Cancer Cell Lines

Cells were plated in a 12-well plate and allowed to adhere overnight. The following day, vehicle or compounds were added to the well in 1 mL of media. After 7-day incubation, medium was replaced with fresh medium containing vehicle or compounds. After 14-day incubation, cells were washed two times in 1×PBS, fixed for 30 minutes in 4% formaldehyde, and then washed twice with 1×PBS. Cells were stained with 0.1% crystal violet for 60 minutes and then washed five times with 1×PBS. Plates were dried for 2 hours at room temperature and imaged. Subsequently, cells were de-stained with 10% acetic acid, and absorbance measured using BCA protocol or absorbance at 560 nm

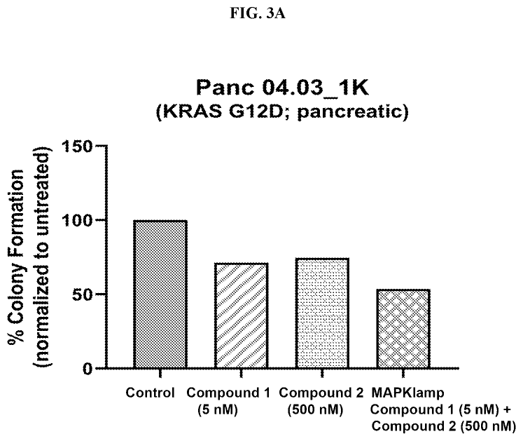

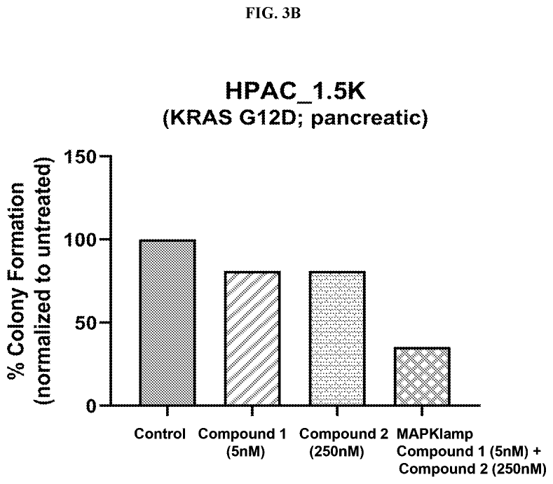

As illustrated by FIG. 3A, Compound 1 and Compound 2 demonstrated a combination benefit in the KRAS G12D mutant pancreatic cell line, Panc 04.03. As illustrated by FIG. 3B, Compound 1 and Compound 2 demonstrate a combination benefit in the KRAS G12D mutant PDAC cell line, HPAC.

### Example 4: In Vivo Studies of Compound 1 Alone, Compound 2 Alone, and Compound 1+Compound 2 Combination in a KRAS G12D Mutant PDAC PDX Model, PAN092

The vehicle/control article, 100 mM acetic acid in deionized water, with pH adjustment to 4.8-5.0, was prepared and stored under ambient conditions throughout the 28-day administration in mice.

The test article Compound 2 was prepared weekly in the vehicle of 100 mM acetic buffer and stored under ambient conditions. The test article Compound 1 was prepared weekly in the vehicle of 0.5% MC and 0.10% Tween 80 solution and stored under ambient conditions throughout the 28-day administration in mice.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation. All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (National Research Council, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of PDX

The PAN092 model was established for nonclinical efficacy studies at GenenDesign (Shanghai, China). The PDX model was derived from a 65-year-old male Chinese PDAC patient that harbored a KRAS G12D mutation. The KRAS G12D mutation in the PDX model PAN092 was confirmed by whole exome sequencing and PCR sequencing. Tumor fragments harvested from the PDX model were implanted subcutaneously in the right flanks of female Balb/c nude mice. Mice were anesthetized with isoflurane, and anesthesia was maintained throughout the implantation procedure. The right flank of the mouse was sterilized with appropriate surgical scrub and alcohol, and aseptic surgical procedures were used. A small skin incision was made using the sharp end of the trocar and a 1.5 cm subcutaneous pocket along the right lateral chest wall was formed by blunt dissection with the stylet of a 10-12 g trocar needle. Tumor fragments (15-30 mm3) were placed into the trocar needle and advanced into the subcutaneous pocket in the right flank. The trocar incision was closed with suture or a wound clip which was removed one week after closure. When tumor sizes reached a mean volume of 200 mm3, tumor-bearing mice were randomly divided into study groups with 8 mice in each group. The randomization date was denoted as treatment day 0.

### Treatment

Treatment started on the day of randomization. The treatment start day was denoted as treatment day 0. Mice were dosed by oral administration of vehicle control solution, Compound 2 at 15 mg/kg QD, and Compound 1 at 30 mg/kg QD, as monotherapies. The Compound 1+Compound 2 treatment group received Compound 1 at 30 mg/kg QD with Compound 2 at 15 mg/kg QD. The dosing volume for each compound was 5 mL/kg. Compound 1 was dosed at one-hour post Compound 2 dose in the Compound 1+Compound 2 treatment group. The study was terminated on treatment day 28 as defined in the study protocol.

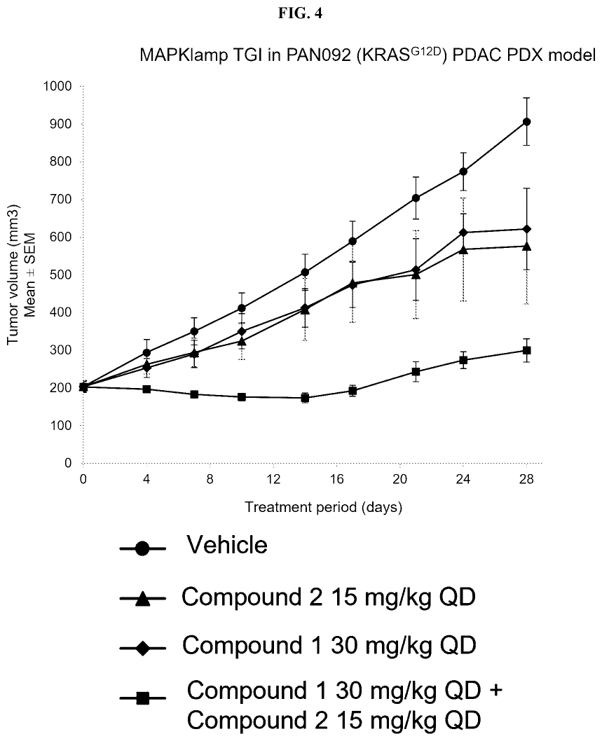

As illustrated by FIG. 4, Compound 1 and Compound 2 demonstrated a combination benefit in vivo in the KRAS G12D mutant PDAC PDX model, PAN092. No significant body weight change was observed in the control and treatment groups.

### Example 5: In Vivo Studies of Compound 1 Alone, Compound 2 Alone, and Compound 1+Compound 2 Combination in a KRAS G12D Mutant PDAC PDX Model, PAN026

The vehicle/control article, 100 mM acetic acid in deionized water, with pH adjustment to 4.8-5.0, was prepared and stored under ambient conditions throughout the 28-day administration in mice.

The test article Compound 2 was prepared weekly in the vehicle of 100 mM acetic buffer and stored under ambient conditions. The test article Compound 1 was prepared weekly in the vehicle of 0.5% MC and 0.1% Tween 80 solution and stored under ambient conditions throughout the 28-day administration in mice.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation. All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (National Research Council, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of PDX

The PAN026 model was established for nonclinical efficacy studies at GenenDesign (Shanghai, China). The PDX model was derived from a 58-year-old male Chinese PDAC patient that harbored a KRAS G12D mutation. The KRAS G12D mutation in the PDX model PAN026 was confirmed by whole exome sequencing and PCR sequencing. Tumor fragments harvested from the PDX model were implanted subcutaneously in the right flanks of female Balb/c nude mice. Mice were anesthetized with isoflurane, and anesthesia was maintained throughout the implantation procedure. The right flank of the mouse was sterilized with appropriate surgical scrub and alcohol, and aseptic surgical procedures were used. A small skin incision was made using the sharp end of the trocar and a 1.5 cm subcutaneous pocket along the right lateral chest wall was formed by blunt dissection with the stylet of a 10-12g trocar needle. Tumor fragments (15-30 mm3) were placed into the trocar needle and advanced into the subcutaneous pocket in the right flank. The trocar incision was closed with suture or a wound clip which was removed one week after closure. When tumor sizes reached a mean volume of 190 mm3, tumor-bearing mice were randomly divided into study groups with 8 mice in each group. The randomization date was denoted as treatment day 0.

### Treatment

Treatment started on the day of randomization. The treatment start day was denoted as treatment day 0. Mice were dosed by oral administration of vehicle control solution, Compound 2 at 15 mg/kg QD, and Compound 1 at 30 mg/kg QD, as monotherapies. The Compound 1+Compound 2 combination treatment group received Compound 1 at 30 mg/kg QD with Compound 2 at 15 mg/kg QD. The dosing volume for each compound was 5 mL/kg. Compound 1 was dosed at one-hour post Compound 2 dose in the Compound 1+Compound 2 combination treatment group. The study was terminated on treatment day 28 as defined in the study protocol.

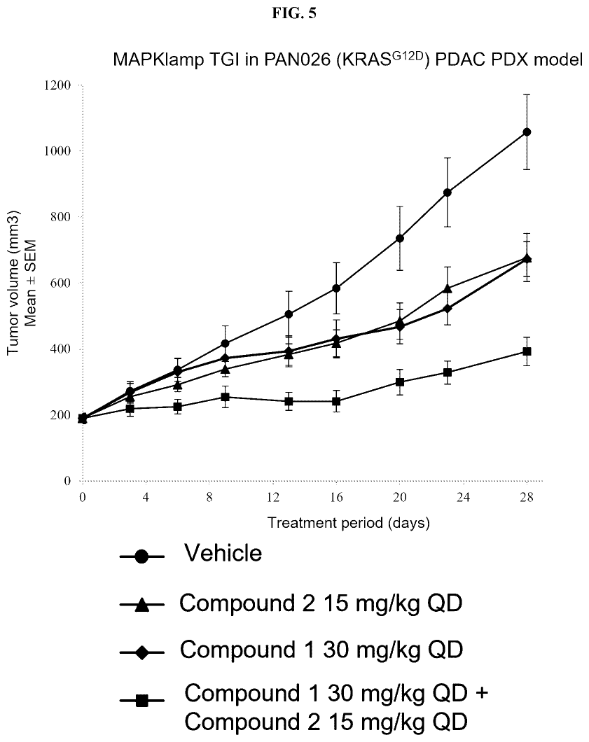

As illustrated by FIG. 5, Compound 1 and Compound 2 demonstrated a combination benefit in vivo in the KRAS G12D mutant PDAC PDX model, PAN026. No significant body weight change was observed in the control and treatment groups.

### Example 6: In Vivo Studies of Compound 1 Alone, Compound 2 Alone, and Compound 1+Compound 2 Combination in a NF1 LoF Mutant Melanoma CDX Model, MeWo

The vehicle/control article, 100 mM acetic acid in deionized water, with pH adjustment to 4.8-5.0, was prepared and stored under ambient conditions throughout the 28-day administration in mice.

The test article Compound 2 was prepared weekly in the vehicle of 100 mM acetic buffer and stored under ambient conditions. The test article Compound 1 was prepared weekly in the vehicle of 0.5% MC and 0.10% Tween 80 solution and stored under ambient conditions throughout the 28-day administration in mice.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation. All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (National Research Council, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of CDX

MeWo was a human melanoma cell line that harbored a NF1LOF mutation (NF1 Q1336*). The MeWo cell line was purchased from the American Type Culture Collection (ATCC® HTB-65™) MeWo cells were cultured in medium containing minimum essential media (MEM) plus 10% fetal bovine serum (FBS) at 37° C. in an atmosphere of 5% CO2 in air. The medium was renewed every 2 to 3 days and tumor cells were routinely sub-cultured at a confluence of 80-90% by trypsin-EDTA. The cells growing in an exponential growth phase were harvested and counted for inoculation. MeWo tumor cells were implanted into mice subcutaneously. 200 μL cell suspensions containing 5×10⁶ tumor cells mixed with 50% Matrigel were subcutaneously implanted into the right flanks of mice using a syringe. Animal health and tumor growth were monitored daily. Tumor volumes were measured twice a week by caliper when tumors were palpable and measurable. When tumor volumes reached a mean of 195 mm3, tumor-bearing mice were randomized into different groups with 8 mice in each group. The randomization date was denoted as treatment day 0.

### Treatment

Treatment started on the day of randomization. The treatment start day was denoted as treatment day 0. Mice were dosed by oral administration of vehicle control solution, Compound 2 at 15 mg/kg QD, and Compound lat 30 mg/kg QD, as monotherapies. The Compound 1+Compound 2 combination treatment group received Compound 1 at 30 mg/kg QD with Compound 2 at 15 mg/kg QD. The dosing volume for each compound was 5 mL/kg. Compound 1 was dosed at one-hour post Compound 2 dose in the Compound 1+Compound 2 combination treatment group. The study was terminated on treatment day 28 as defined in the study protocol.

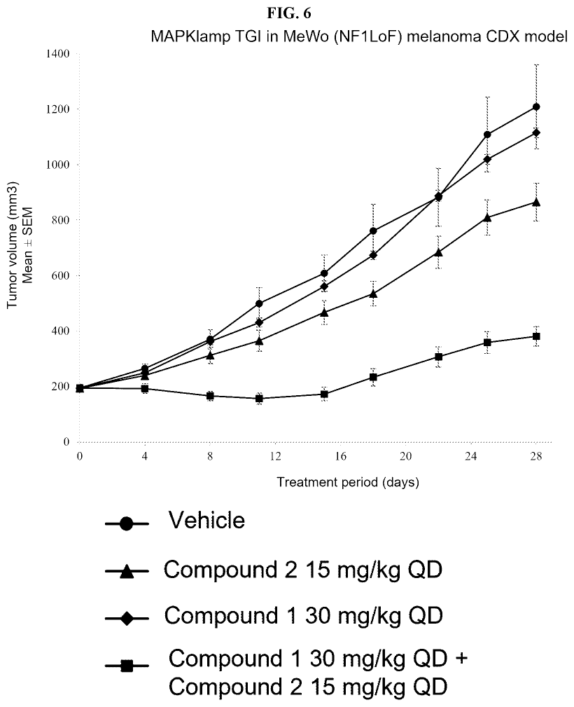

As illustrated by FIG. 6, Compound 1 and Compound 2 demonstrated a combination benefit in vivo in the NF1 LoF mutant melanoma CDX model, MeWo. No significant body weight change was observed in the control and treatment groups.

### Example 7: In Vivo Studies of Compound 1 Alone, Compound 2 Alone, and Compound 1+Compound 2 Combination in a KRAS G13D Mutant CRC CDX Model, LoVo

The vehicle/control article, 100 mM acetic acid in deionized water, with pH adjustment to 4.8-5.0, was prepared and stored under ambient conditions throughout the 28-day administration in mice.

The test article Compound 2 was prepared weekly in the vehicle of 100 mM acetic buffer and stored under ambient conditions. The test article Compound 1 was prepared weekly in the vehicle of 0.5% MC and 0.10% Tween 80 solution and stored under ambient conditions throughout the 28-day administration in mice.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation. All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (National Research Council, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of CDX

LoVo is a human CRC cell line harboring a KRAS G13D mutation. The LoVo cell line was purchased from the American Type Culture Collection (ATCC® CCL-229™). LoVo cells were cultured in F12K medium containing 10% fetal bovine serum (FBS) at 37° C. in an atmosphere of 5% CO2 in air. The medium was renewed every 2 to 3 days and tumor cells were routinely sub-cultured at a confluency of 80-90%. The cells growing in an exponential growth phase were harvested using trypsin-EDTA, counted for inoculation, and subsequently implanted into mice subcutaneously. 200 μL cell suspensions containing 2×10⁶ tumor cells mixed with 50% Matrigel were subcutaneously implanted into the right flanks of mice using a syringe. Animal health and tumor growth were monitored daily. Tumor volumes were measured twice a week by caliper when tumors were palpable and measurable. When tumor volumes reached a mean of 190 mm3, tumor-bearing mice were randomized into different groups with 8 mice in each group. The randomization date was denoted as treatment day 0.

### Treatment

Treatment started on the day of randomization. The treatment start day was denoted as treatment day 0. Mice were dosed by oral administration of vehicle control solution, Compound 2 at 15 mg/kg QD, and Compound 1 at 30 mg/kg QD, as monotherapies. The Compound 1+Compound 2 combination treatment group received Compound 1 at 30 mg/kg QD with Compound 2 at 15 mg/kg QD. The dosing volume for each compound was 5 mL/kg. Compound 1 was dosed at one-hour post Compound 2 dose in the Compound 1+Compound 2 combination treatment group. The study was terminated on treatment day 28 as defined in the study protocol.

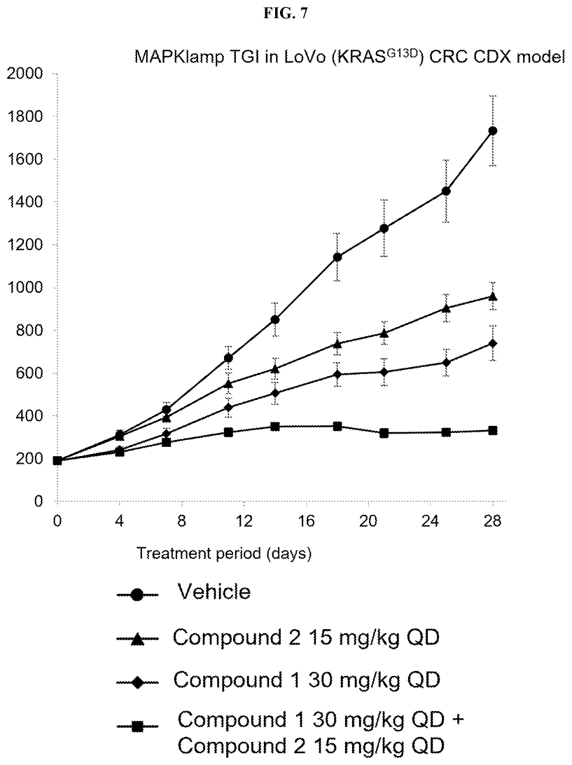

As illustrated by FIG. 7 Compound 1 and Compound 2 demonstrated a combination benefit in vivo in the KRAS G13D mutant CRC CDX model, LoVo. No significant body weight change was observed in the control and treatment groups.

### Example 8—In Vitro Studies of Compound 1 Alone, Compound 2 Alone, and Compound 1+Compound 2 Combination in KRAS G12V Mutant PDAC Cell Line, Capan-2, in 14 Days Clonogenic Assay

Capan-2 cells were plated at 4000 cells/well density in a 12-well plate and allowed to adhere overnight (see table below). The following day, compounds were added to the well in 1 mL of media. After 7-day incubation, medium was replaced with fresh medium containing vehicle or compounds. After 14-day incubation, cells were washed two times in 1×PBS, fixed for 30 minutes in 4% formaldehyde, and then washed twice with 1×PBS. Cells were stained with 0.10% crystal violet for 60 minutes and then washed five times with 1×PBS. Plates were dried for 2 hours at room temperature and imaged. Subsequently, cells were de-stained with 10% acetic acid, and absorbance measured using BCA protocol or absorbance at 560 nm.

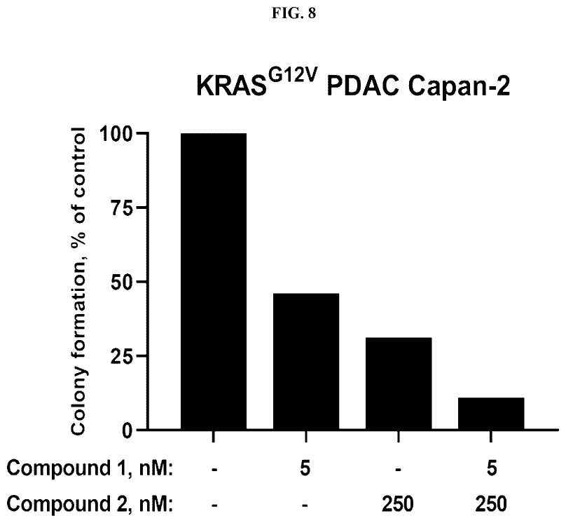

As illustrated by FIG. 8, Compound 1 and Compound 2 demonstrated a combination benefit in the KRAS G12V mutant PDAC cell line, Capan-2.

### Example 9—In Vitro Studies of Compound 1 Alone, Compound 2 Alone, and Compound 1+Compound 2 Combination in KRAS G12V Mutant PDAC Cell Line, Panc 10.05, in 14 Days Clonogenic Assay

Panc 10.05 cells were plated at 2000 cells/well density in a 12-well plate and allowed to adhere overnight (see table below). The following day, compounds were added to the well in 1 mL of media. After 7-day incubation, medium was replaced with fresh medium containing vehicle or compounds. After 14-day incubation, cells were washed two times in 1×PBS, fixed for 30 minutes in 4% formaldehyde, and then washed twice with 1×PBS. Cells were stained with 0.10% crystal violet for 60 minutes and then washed five times with 1×PBS. Plates were dried for 2 hours at room temperature and imaged. Subsequently, cells were de-stained with 10% acetic acid, and absorbance measured using BCA protocol or absorbance at 560 nm.

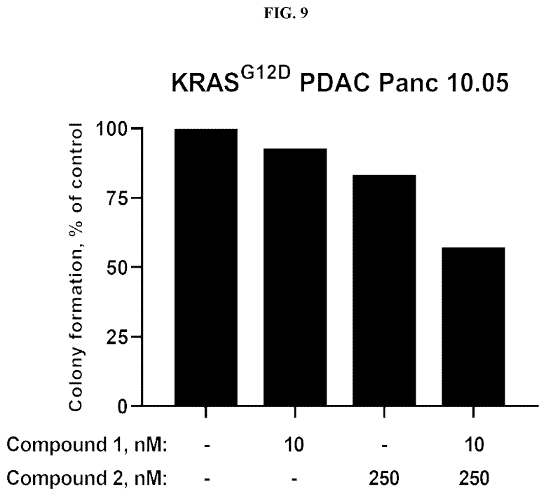

As illustrated by FIG. 9, Compound 1 and Compound 2 demonstrated a combination benefit in the KRAS G12V mutant PDAC cell line, Panc 10.05.

### Example 10—In Vitro Studies of Compound 1 Alone, Compound 2 Alone, and Compound 1+Compound 2 Combination in KRAS G12V Mutant PDAC Cell Line, Panc 1, in 14 Days Clonogenic Assay

Panc-1 cells were plated at 4000 cells/well density in a 12-well plate and allowed to adhere overnight (see table below). The following day, compounds were added to the well in 1 mL of media. After 7-day incubation, medium was replaced with fresh medium containing vehicle or compounds. After 14-day incubation, cells were washed two times in 1×PBS, fixed for 30 minutes in 4% formaldehyde, and then washed twice with 1×PBS. Cells were stained with 0.10% crystal violet for 60 minutes and then washed five times with 1×PBS. Plates were dried for 2 hours at room temperature and imaged. Subsequently, cells were de-stained with 10% acetic acid, and absorbance measured using BCA protocol or absorbance at 560 nm.

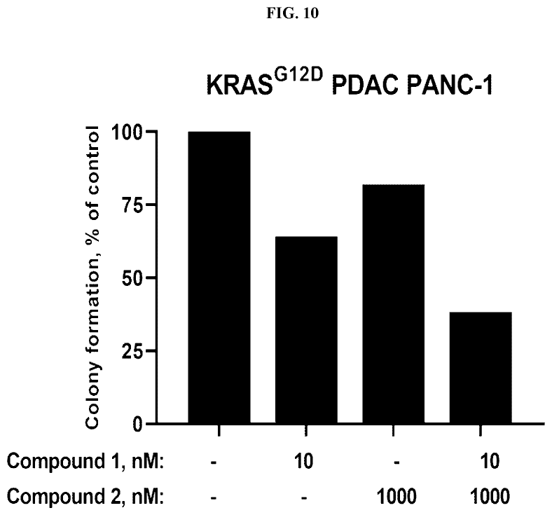

As illustrated by FIG. 10, Compound 1 and Compound 2 demonstrated a combination benefit in the KRAS G12V mutant PDAC cell line, Panc 1.

1. A method of treating cancer in a subject in need thereof, the method comprising: administering to the subject in need thereof a therapeutically effective amount of
(i) compound 1: 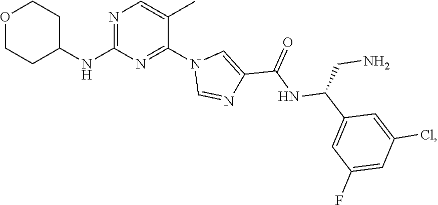 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) a SHP2 inhibitor.

2. The method of claim 1, wherein the SHP2 inhibitor is sodium stibogluconate, RMC-4550, NSC87877, SPI-112, TNO155, IACS-13909, SHP099 HCl, or compound 2: 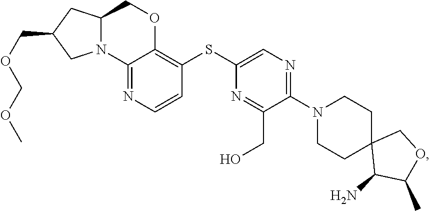 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof.

3. A method of treating cancer in a subject in need thereof, the method comprising: administering to the subject in need thereof a therapeutically effective amount of
(i) compound 1: 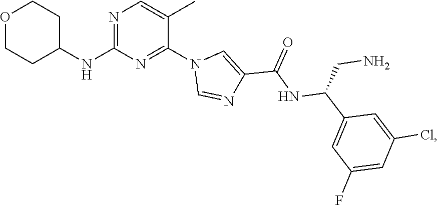 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) compound 2: 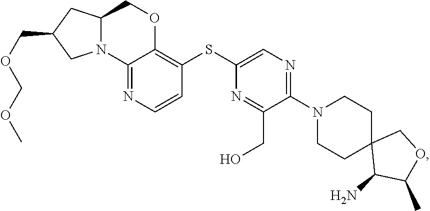 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof.

4. A method of treating cancer in a subject in need thereof, the method comprising: administering to the subject in need thereof a therapeutically effective amount of
(i) compound 1: 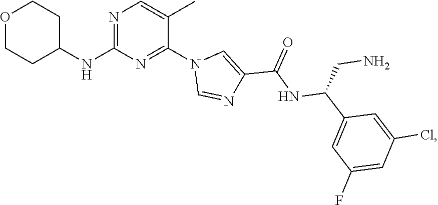 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 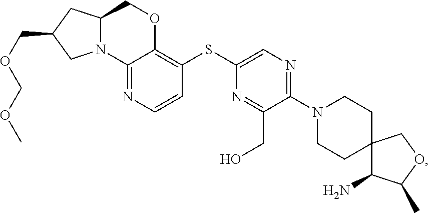 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) osimertinib.

5. The method of claim 4, wherein osimertinib is administered in an amount that is about 80 mg/day.

6. A method of treating cancer in a subject in need thereof, the method comprising: administering to the subject in need thereof a therapeutically effective amount of
(i) compound 1: 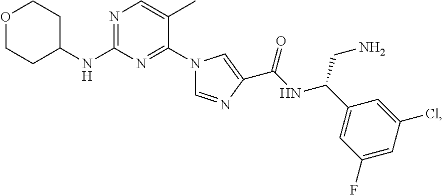 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 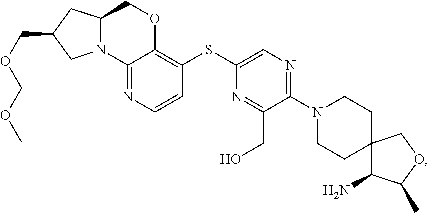 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) cetuximab.

7. The method of claim 6, wherein cetuximab is administered at 500 mg/m² once every two weeks, 400 mg/m2 once every two weeks, or 300 mg/m² once every two weeks.

8. A method of treating cancer in a subject in need thereof, the method comprising: administering to the subject in need thereof a therapeutically effective amount of
(i) compound 1: 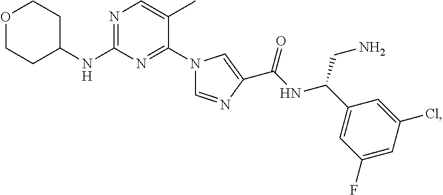 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 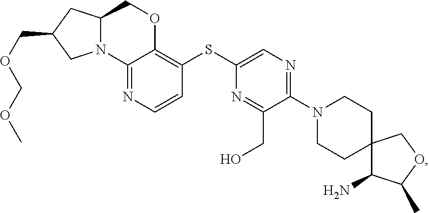 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof;
(iii) encorafenib; and
(iv) osimertinib.

9. The method of claim 8, wherein encorafenib is administered in an amount that is between about 100 mg/day and about 500 mg/day.

10. The method of claim 8 or 9, wherein encorafenib is administered in an amount that is about 450 mg/day, 300 mg/day, 225 mg/day, or 150 mg/day.

11. The method of any one of claims 8 to 10, wherein osimertinib is administered at about 80 mg/day.

12. A method of treating cancer in a subject in need thereof, the method comprising: administering to the subject in need thereof a therapeutically effective amount of
(i) compound 1: 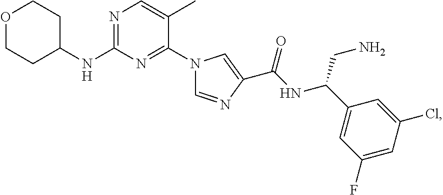 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 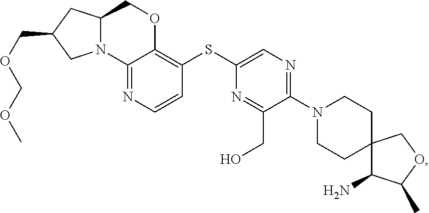 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof;
(iii) encorafenib; and
(iv) cetuximab.

13. The method of claim 12, wherein encorafenib is administered in an amount that is between about 100 mg/day and about 500 mg/day.

14. The method of claim 12 or 13, wherein encorafenib is administered in an amount that is about 450 mg/day, about 300 mg/day, about 225 mg/day, or about 150 mg/day.

15. The method of any one of claims 12 to 14, wherein cetuximab is administered at 500 mg/m² once every two weeks, 400 mg/m² once every two weeks, or 300 mg/m² once every two weeks.

16. A method of treating cancer in a subject in need thereof, the method comprising: administering to the subject in need thereof a therapeutically effective amount of
(i) compound 1: 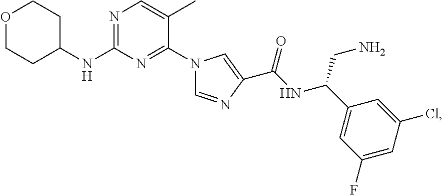 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 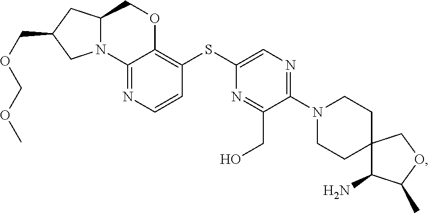 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) gilteritinib.

17. The method of claim 16, wherein gilteritinib is administered in an amount that is about 120 mg/day.

18. A method of treating cancer in a subject in need thereof, the method comprising: administering to the subject in need thereof a therapeutically effective amount of
(i) compound 1: 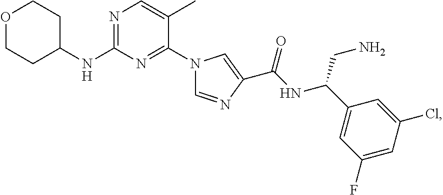 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 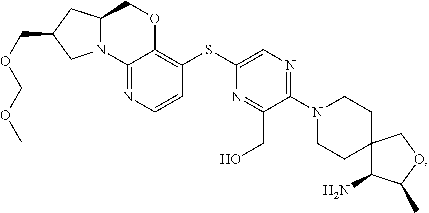 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) sotorasib.

19. The method of claim 18, wherein sotorasib is administered in an amount that is about 960 mg/day.

20. A method of treating cancer in a subject in need thereof, the method comprising: administering to the subject in need thereof a therapeutically effective amount of
(i) compound 1: 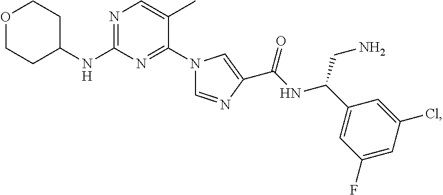 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 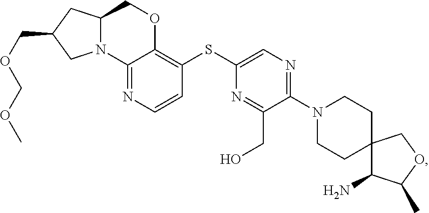 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) adagrasib.

21. The method of claim 20, wherein adagrasib is administered in an amount that is about 1200 mg/day.

22. A method of treating cancer in a subject in need thereof, the method comprising: administering to the subject in need thereof a therapeutically effective amount of
(i) compound 1: 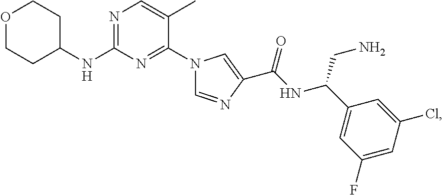 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 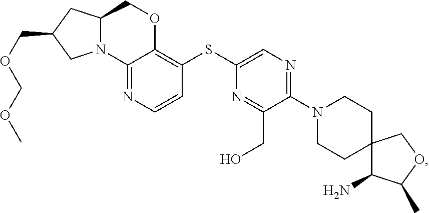 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) palbociclib.

23. The method of claim 22, wherein palbociclib is administered in an amount that is between about 50 mg/day and about 500 mg/day.

24. The method of claim 22 or 23, wherein palbociclib is administered in an amount that is about 75 mg/day, about 100 mg/day, about 125 mg/day, or about 150 mg/day.

25. The method of claim 22, wherein palbociclib is administered in an amount that is between about 50 mg once a week and about 650 mg once a week.

26. The method of claim 22 or 23, wherein palbociclib is administered in an amount that is about 200 mg once a week, 300 mg once a week, 400 mg once a week, 500 mg once a week, or 600 mg once a week.

27. The method of any one of claims 2-26, wherein compound 2, or a pharmaceutically acceptable salt thereof, is administered orally.

28. The method of any one of claims 2-27, wherein compound 2, or a pharmaceutically acceptable salt thereof, is administered in an amount that is between about 1 mg/day and about 500 mg/day.

29. The method of any one of claims 2-27, wherein compound 2, or a pharmaceutically acceptable salt thereof, is administered in an amount that is between about 20 mg/day and about 400 mg/day.

30. The method of any one of claims 2-27, wherein compound 2, or a pharmaceutically acceptable salt thereof, is administered in an amount that is between about 30 mg/day and about 300 mg/day.

31. The method of any one of claims 2-27, wherein compound 2, or a pharmaceutically acceptable salt thereof, is administered QD or BID for 2 weeks on and 1 week off (21 day schedule).

32. The method of any one of claims 2-27, wherein compound 2, or a pharmaceutically acceptable salt thereof, is administered QD or BID for 3 weeks on and 1 week off (28 day schedule).

33. The method of any one of claims 2-27, wherein compound 2, or a pharmaceutically acceptable salt thereof, is administered QD or BID three times a week (D1D3D5 TIW) e.g., Day 1, Day 3, and Day 5.

34. The method of any one of claims 2-27, wherein compound 2, or a pharmaceutically acceptable salt thereof, is administered twice a day/twice a week e.g., Day 1 and Day 2 (BID-D1D2-BIW).

35. The method of any one of claims 2-27, wherein compound 2, or a pharmaceutically acceptable salt thereof, is administered once a day (QD) continuous dosing at a dose of 20 mg/day to 60 mg/day, 40 mg/day, or 60 mg/day.

36. The method of any one of claims 2-27, wherein compound 2, or a pharmaceutically acceptable salt thereof, is administered twice a day (BID) continuous dosing at a dose of 20 mg/day to 80 mg/day.

37. The method of any one of claims 2-27, wherein compound 2, or a pharmaceutically acceptable salt thereof, is administered twice a day (BID) continuous dosing at a dose of 10 mg/day to 100 mg/day.

38. The method of any one of claims 1-37, wherein the pharmaceutically acceptable salt of compound 1 is the mandelic acid salt.

39. The method of any one of claims 1-38, wherein the cancer is a mitogen-activated protein kinase (MAPK) pathway driven cancer.

40. The method of any one of claims 1-38, wherein the cancer is a BRAF-driven cancer, HRAS-driven cancer, or a NRAS-driven cancer.

41. The method of any one of claims 1-38, wherein the cancer comprises at least one cancer cell driven by deregulated ERK.

42. The method of any one of claims 1-38, wherein the cancer has at least one mutation in RAS.

43. The method of any one of claims 1-38, wherein the cancer has at least one mutation in RAF.

44. The method of any one of claims 1-38, wherein the cancer has at least one mutation in MEK.

45. The method of any one of claims 1-38, wherein the cancer has a G12C KRAS mutation.

46. The method of any one of claims 1-38, wherein the cancer has a G12D KRAS mutation.

47. The method of any one of claims 1-38, wherein the cancer has a G12S KRAS mutation.

48. The method of any one of claims 1-38, wherein the cancer has a G12V KRAS mutation.

49. The method of any one of claims 1-38, wherein the cancer has a G13D KRAS mutation.

50. The method of any one of claims 1-38, wherein the cancer has a Q16H KRAS mutation.

51. The method of any one of claims 1-38, wherein the cancer has a Q16K KRAS mutation.

52. The method of any one of claims 1-38, wherein the cancer has a Q61R NRAS mutation.

53. The method of any one of claims 1-38, wherein the cancer is a BRAF V600E or V600K mutant tumor.

54. The method of any one of claims 1-38, wherein the cancer is a MAPKm/MAPKi-naïve pancreatic cancer or PDAC.

55. The method of any one of claims 1-38, wherein the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

56. The method of any one of claims 1-38, wherein the cancer comprises one or more EGFR mutation selected from the group consisting of L858R, exon 19 deletion, and T790M.

57. The method of any one of claims 1-56, wherein the cancer is a liquid tumor.

58. The method of claim 57, wherein the liquid tumor is leukemia.

59. The method of claim 57, wherein the leukemia is acute myeloid leukemia (AML).

60. The method of claim 59, wherein the AML is relapsed and/or refractory AML.

61. The method of claim 59, wherein the AML is a FLT3 mutant AML.

62. The method of any one of claims 1-56, wherein the cancer is a solid tumor.

63. The method of any one of claims 1-56 or 62, wherein the cancer is non-small cell lung cancer (NSCLC), melanoma, pancreatic cancer, salivary gland tumor, thyroid cancer, colorectal cancer (CRC), or esophageal cancer.

64. The method of any one of claims 1-56 or 62, wherein the cancer is non-small cell lung cancer (NSCLC).

65. The method of claim 64, wherein the NSCLC is an EGFR mutant NSCLC.

66. The method of claim 64, wherein the NSCLC is a KRAS G12C mutant NSCLC.

67. The method of claim 64, wherein the NSCLC is a KRAS G12D mutant NSCLC.

68. The method of claim 64, wherein the NSCLC is a KRAS G12S mutant NSCLC.

69. The method of claim 64, wherein the NSCLC is a KRAS G12V mutant NSCLC.

70. The method of claim 64, wherein the NSCLC is a KRAS G12A mutant NSCLC.

71. The method of claim 64, wherein the NSCLC is a KRAS G13D mutant NSCLC.

72. The method of claim 64, wherein the NSCLC is a KRAS Q61H mutant NSCLC.

73. The method of claim 64, wherein the NSCLC is a KRAS Q61K mutant NSCLC.

74. The method of claim 64, wherein the NSCLC is a NRAS Q61R mutant NSCLC.

75. The method of claim 64, wherein the cancer is a MAPKm/MAPKi-naïve NSCLC.

76. The method of claim 64, wherein the cancer is a BRAFi-treated V600 NSCLC.

77. The method of claim 64, wherein the cancer is a KRAS-treated G12C NSCLC.

78. The method of claim 64, wherein the cancer is a KRAS-treated G12D NSCLC.

79. The method of claim 64, wherein the cancer is a KRAS-treated G12S NSCLC.

80. The method of claim 64, wherein the cancer is a KRAS-treated G12V NSCLC.

81. The method of claim 64, wherein the cancer is a KRAS-treated G13D NSCLC.

82. The method of claim 64, wherein the cancer is a KRAS-treated Q61H NSCLC.

83. The method of claim 64, wherein the cancer is a KRAS-treated Q61K NSCLC.

84. The method of claim 64, wherein the cancer is a NRAS-treated Q61R NSCLC.

85. The method of any one of claims 1-56 or 62, wherein the cancer is pancreatic cancer.

86. The method of any one of claims 1-56 or 62, wherein the cancer is a MAPKm/MAPKi-naïve pancreatic cancer.

87. The method of claim 85, wherein the pancreatic cancer is pancreatic ductal adenocarcinoma (PDAC).

88. The method of any one of claims 1-56 or 62, wherein the cancer is melanoma.

89. The method of claim 88, wherein the melanoma has an NF1 Loss of Function (NF1-LoF) mutation.

90. The method of claim 88, wherein the melanoma is a BRAF V600E or V600K mutant tumor.

91. The method of claim 88, wherein the cancer is a BRAFi-treated V600 melanoma.

92. The method of any one of claims 1-56 or 62, wherein the cancer is salivary gland tumor.

93. The method of any one of claims 1-56 or 62, wherein the cancer is thyroid cancer.

94. The method of any one of claims 1-56 or 62, wherein the cancer is colorectal cancer (CRC).

95. The method of claim 94, wherein the CRC is a BRAF V600E CRC.

96. The method of claim 94, wherein the CRC is a KRAS mutant CRC.

97. The method of claim 94, wherein the CRC is a KRAS G12C mutant CRC.

98. The method of claim 94, wherein the CRC is a KRAS G12D mutant CRC.

99. The method of claim 94, wherein the CRC is a KRAS G12S mutant CRC.

100. The method of claim 94, wherein the CRC is a KRAS G12V mutant CRC.

101. The method of claim 94, wherein the CRC is a KRAS G13D mutant CRC.

102. The method of claim 94, wherein the CRC is a KRAS Q61H mutant CRC.

103. The method of claim 94, wherein the CRC is a KRAS Q61K mutant CRC.

104. The method of claim 94, wherein the CRC is a NRAS mutant CRC.

105. The method of claim 94, wherein the CRC is NRAS Q61R mutant CRC.

106. The method of any one of claims 1-56 or 62, wherein the cancer is esophageal cancer.

107. The method of any one of claims 1-106, wherein compound 1, or a pharmaceutically acceptable salt thereof, is administered in an amount that is between about 25 mg/day and about 300 mg/day.

108. The method of any one of claims 1-107, wherein compound 1, or a pharmaceutically acceptable salt thereof, is administered in an amount that is between 25 mg/day and 150 mg/day.

109. The method of any one of claims 1-108, wherein compound 1, or a pharmaceutically acceptable salt thereof, is administered in an amount that is about 25 mg/day, about 50 mg/day, about 75 mg/day, about 100 mg/day, about 150 mg/day, about 175 mg/day, about 200 mg/day, about 225 mg/day, or about 250 mg/day.

110. The method of any one of claims 1-109, wherein compound 1, or a pharmaceutically acceptable salt thereof, is administered in an amount that is about 25 mg/day, about 50 mg/day, about 100 mg/day, or about 150 mg/day.

111. The method of any one of claims 1-109, wherein compound 1, or a pharmaceutically acceptable salt thereof, is administered in an amount that is about 250 mg/day.

112. The method of any one of claims 1-111, wherein compound 1, or a pharmaceutically acceptable salt thereof, is administered once a day (QD).

113. The method of any one of claims 1-111, wherein compound 1, or a pharmaceutically acceptable salt thereof, is administered twice a day (BID).

114. The method of any one of claims 1-111, wherein compound 1, or a pharmaceutically acceptable salt thereof, is administered three times a day (TID).

115. The method of any one of claims 1-114, wherein compound 1, or a pharmaceutically acceptable salt thereof, is administered once a week.

116. The method of any one of claims 1-114, wherein compound 1, or a pharmaceutically acceptable salt thereof, is administered twice a week.

117. The method of any one of claims 1-106, wherein compound 1, or a pharmaceutically acceptable salt thereof, is administered in an amount that is between about 25 mg and about 300 mg twice a day, once a week (BID-QW).

118. The method of any one of claims 1-106, wherein compound 1, or a pharmaceutically acceptable salt thereof, is administered in an amount that is between about 25 mg and about 250 mg twice a day, once a week (BID-QW).

119. The method of any one of claims 1-106, wherein compound 1, or a pharmaceutically acceptable salt thereof, is administered in an amount that is between about 25 mg and about 150 mg twice a day, once a week (BID-QW).

120. The method of any one of claims 1-106, wherein compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

121. The method of any one of claims 1-106, wherein compound 1, or a pharmaceutically acceptable salt thereof, is administered in an amount that is about 25 mg, 50 mg, about 100 mg, about 125 mg, or about 150 mg twice a day, once a week (BID-QW).

122. The method of any one of claims 1-106, wherein compound 1, or a pharmaceutically acceptable salt thereof, is administered in an amount that is about 125 mg twice a day, once a week (BID-QW).

123. The method of any one of claims 1-122, wherein compound 1, or a pharmaceutically acceptable salt thereof, is administered for at least one 28-day cycle.

124. The method of any one of claims 1-123, wherein compound 1, or a pharmaceutically acceptable salt thereof, is administered on day 1, day 8, day 15, and day 22 of a 28-day cycle.

125. The method of any one of claims 1-123, wherein compound 1, or a pharmaceutically acceptable salt thereof, is administered on day 1, day 8, day 15 of a 28-day cycle.

126. The method of any one of claims 1-125, wherein compound 1, or a pharmaceutically acceptable salt thereof, is administered orally.

127. The method of any one of claims 1-126, wherein the method further comprises administering an additional MAPK pathway inhibitor.

128. The method of claim 127, wherein the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor.

129. The method of claim 127, wherein the additional MAPK pathway inhibitor is a KRAS inhibitor.

130. The method of claim 127, wherein the additional MAPK pathway inhibitor is a BRAF inhibitor.

131. The method of claim 127, wherein the additional MAPK pathway inhibitor is an EGFR inhibitor.

132. The method of claim 127, wherein the additional MAPK pathway inhibitor is a CDK4/6.

133. The method of claim 127, wherein the additional MAPK pathway inhibitor is a FLT3 inhibitor.

134. The method of claim 127, wherein the additional MAPK pathway inhibitor is adagrasib, afatinib, ASTX029, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, panitumumab, selumetinib, sotorasib, trametinib, ulixertinib, vandetanib, or vemurafenib.

135. The method of claim 127, wherein the additional MAPK pathway inhibitor is adagrasib.

136. The method of claim 127, wherein the additional MAPK pathway inhibitor is cetuximab.

137. The method of claim 127, wherein the additional MAPK pathway inhibitor is dabrafenib.

138. The method of claim 127, wherein the additional MAPK pathway inhibitor is encorafenib.

139. The method of claim 127, wherein the additional MAPK pathway inhibitor is gilteritinib.

140. The method of claim 127, wherein the additional MAPK pathway inhibitor is palbociclib.

141. The method of claim 127, wherein the additional MAPK pathway inhibitor is panitumumab.

142. The method of claim 127, wherein the additional MAPK pathway inhibitor is sotorasib.
